SEQUENCE LISTING (30 sequences)

SEQ ID NO: 1 - DNA, 29 bp, Artificial Sequence
  Forward primer
ggtggttcccaataaaagtgactctcagc

SEQ ID NO: 2 - DNA, 35 bp, Artificial Sequence
  Forward primer rA
ggtggttcccaataaaagtgactctcagcatgcct

SEQ ID NO: 3 - DNA, 35 bp, Artificial Sequence
  Forward primer rG
ggtggttcccaataaaagtgactctcagcgtgcct

SEQ ID NO: 4 - DNA, 51 bp, Homo sapiens
ggtggttcccaataaaagtgactctcagcgagcctcaatgctcccagtgct

SEQ ID NO: 5 - DNA, 40 bp, Artificial Sequence
  Complement of reverse primer Tail 1
tggtagacgctctaatgctctcgtgaccctcgtaactccg

SEQ ID NO: 6 - DNA, 40 bp, Artificial Sequence
  Reverse primer Tail 1
cggagttacgagggtcacgagagcattagagcgtctacca

SEQ ID NO: 7 - DNA, 40 bp, Artificial Sequence
  Complement of reverse primer Tail 2
ctaggatcgcgagtgcataatcgtgaccctcgtaactccg

SEQ ID NO: 8 - DNA, 40 bp, Artificial Sequence
  Reverse primer Tail 2
cggagttacgagggtcacgattatgcactcgcgatcctag

SEQ ID NO: 9 - DNA, 24 bp, Artificial Sequence
  Forward primer
cggggagggagctgaccagtgaag

SEQ ID NO: 10 - DNA, 31 bp, Artificial Sequence
  Forward primer rA
cggggagggagctgaccagtgaagatagtgt

SEQ ID NO: 11 - DNA, 31 bp, Artificial Sequence
  Forward primer rG
cggggagggagctgaccagtgaagctagtgt

SEQ ID NO: 12 - DNA, 54 bp, Homo sapiens
cggggagggagctgaccagtgaagcaagtgtgctttgaagtctttgttcttacc

SEQ ID NO: 13 - DNA, 48 bp, Artificial Sequence
  Compleemnt of reverse primer Tail 1
tggtagacgctctaatgctcccattcttgtttctgaagtttcgtgtga

SEQ ID NO: 14 - DNA, 48 bp, Artificial Sequence
  Reverse primer Tail 1
tcacacgaaacttcagaaacaagaatgggagcattagagcgtctacca

SEQ ID NO: 15 - DNA, 48 bp, Artificial Sequence
  Compleemnt of reverse primer Tail 2
ctaggatcgcgagtgcataaccattcttgtttctgaagtttcgtgtga

SEQ ID NO: 16 - DNA, 48 bp, Artificial Sequence
  Reverse primer Tail 2
tcacacgaaacttcagaaacaagaatggttatgcactcgcgatcctag

SEQ ID NO: 17 - DNA, 20 bp, Artificial Sequence
  Forward primer
gagcagatccctggacaggc

SEQ ID NO: 18 - DNA, 26 bp, Artificial Sequence
  Forward primer rA
gagcagatccctggacaggcatggaa

SEQ ID NO: 19 - DNA, 26 bp, Artificial Sequence
  Forward primer rG
gagcagatccctggacaggcgtggaa

SEQ ID NO: 20 - DNA, 45 bp, Artificial Sequence
  Reverse primer Tail 1
accatcgtcgagattacgtcttcaaggacaaaatacctgtattcc

SEQ ID NO: 21 - DNA, 45 bp, Artificial Sequence
  Reverse primer Tail 2
gatcctaggcctcacgtattttcaaggacaaaatacctgtattcc

SEQ ID NO: 22 - DNA, 27 bp, Artificial Sequence
  Forward primer
gccgcccactgtttggctttcagttat

SEQ ID NO: 23 - DNA, 34 bp, Artificial Sequence
  Forward primer rM
gccgcccactgtttggctttcagttataaggatg

SEQ ID NO: 24 - DNA, 35 bp, Artificial Sequence
  Forward primer rV
gccgcccactgtttggctttcagttatgaggatga

SEQ ID NO: 25 - DNA, 36 bp, Artificial Sequence
  Forward primer rI
gccgcccactgtttggctttcagttataatgatgat

SEQ ID NO: 26 - DNA, 46 bp, Hepatitis B virus
cccactgtttggctttcagttatatggatgatgtggtattgggggc

SEQ ID NO: 27 - DNA, 40 bp, Artificial Sequence
  Complement of reverse primer Tail 1
tggtagacgctctaatgctccgggggttatggtgtagtag

SEQ ID NO: 28 - DNA, 40 bp, Artificial Sequence
  Reverse primer Tail 1
ctactacaccataacccccggagcattagagcgtctacca

SEQ ID NO: 29 - DNA, 40 bp, Artificial Sequence
  Complement of reverse primer Tail 2
ctaggatcgcgagtgcaatacgggggttatggtgtagtag

SEQ ID NO: 30 - DNA, 40 bp, Artificial Sequence
  Reverse primer Tail 2
ctactacaccataacccccgtattgcactcgcgatcctag

(19) United States
(12) Patent Application Publication
(10) Pub. No.: US 2005/0106575 A1
(43) Pub. Date: May 19, 2005

(54) Method and kit for detecting mutation or nucleotide variation of organism

(75) Inventors: Xianqiang Li, Palo Alto, CA (US); Takuro Yaoi, Fremont, CA (US)

Correspondence Address: WILSON SONSINI GOODRICH & ROSATI, 650 PAGE MILL ROAD, PALO ALTO, CA, 943041050, US

(73) Assignee: Panomics, Inc., Redwood City, CA (US)

(21) Appl. No.: 10/714,579

(22) Filed: Nov. 13, 2003

(51) Int. Cl. C12Q 1/70; C12Q 1/68; C12P 19/34

(52) U.S. Cl. 435/6; 435/5; 435/91.2; 257/40

(57) ABSTRACT

The present invention provides methods, compositions and kits for highly efficient, high throughput detection of mutation or nucleotide variation of an organism. By exploiting the molecular interactions between strands of nucleic acid and between nucleic acid and protein, assays have been developed to detect nucleotide variation, in particular, single nucleotide polymorphism (SNP) in various biological samples including human genomic DNA and virus. In preferred embodiments, immunoassays are developed to specifically capture a nucleic acid-protein complex formed between a 4-way nucleic acid structure called Holliday junction and a protein that specifically recognizes the Holliday junction. These assays can be used in a wide variety of applications such as diagnostics, genotyping, genetic profiling, mutation detection, disease prevention, therapeutic treatment, and screening for therapeutic targets or therapeutics.

### FIELD OF INVENTION

The present invention relates to methods, compositions and kits for detecting mutation or variation of nucleotide of an organism, and in particular, relates to high throughput immunoassays for detecting mutation or single nucleotide polymorphism in genomic DNA of an organism such as human virus, or for genotyping and allele frequency determination of an organism such as human.

### BACKGROUND OF THE INVENTION

Single-nucleotide polymorphisms (SNPs) are common individual variations, which are found every 250-350 bp and are responsible for the majority of genetic variation between human beings. Cargill et al. (1999) Nat. Genet. 22: 231. SNPs are single nucleotides among the DNA sequence at which two alternative bases (diallelic polymorphisms) occur at appreciable frequency (>1%) in the human population. Human genetic variations (mutations or polymorphisms) result from DNA mutations that may or may not have functional consequences. Approximately 20% of DNA polymorphisms are length polymorphisms, and the remaining 80% are SNPs.

Both genomic and mitochondrial DNA contain large numbers of SNPs. Some SNPs may be involved in a disease process, while the majority probably are not. Because neutral DNA variants are not under selective pressures, they occur at variable frequencies within populations as a result of genetic drift. The current issue is to identify, validate, and map SNPs on the human genome (mutation scanning or screening techniques (DeFrancesco and Perkel (2001) The Scientist 2801077-82), and then use these maps of ordered SNPs for genetic analysis.

SNPs can be used as markers in whole genome linkage analysis of families or in association studies of individuals in a population. The human genome project, and associated efforts to identify SNPs, will give rise to hundreds of thousands of potentially informative, mapped polymorphic sites. Mutation analysis, allied with basic biological studies to link mutation with phenotype in man and model organisms, is one of the fundamental goals of global intiatives in functional genomics. The requirement in population screening is for cheap and effective means to detect previously-identified mutations.

Various methods have developed for detecting SNP. PCR-based methods, with the goal of providing practical and inexpensive assays for SNP detection, include oligonucleotide ligation assay (Baron et al. (1996) Nat. Biotechnol. 14:1279-82; and Delahunty et al. (1996) A. J. Hum. Genet. 58:1239-46), allele-specific amplification (Wu et al. (1989) Proc. Natl. Acad. Sci. 86:2757-60), allele-specific oligonucleotide hybridization (Saiki et al. (1989) Proc. Natl. Acad. Sci. 86:6230-4), mini-sequencing (Ahmadian et al. (2000) Anal. Biochem. 280:102-10; and Alderborn et al. (2000) Genome Res. 10:1249-58), homogeneous proximity assays (Beaudet et al. (2001) Genome Res. 11:600-8), high-density oligonucleotide probe arrays (Gerry et al. (1999) J. Mol. Biol. 22:252-62; and Murry et al. (2001) Proc. Natl. Acad. Sci. 98:9853-8), and peptide nucleic acid (PNA) hybridization (Ross et al. (1997) Anal. Chem. 69:4197-202). Besides gel electrophoretic analysis, other methods for SNP detection include plate readers, reverse dot-blot hybridization and mass spectrometric detection. Some of these detection methods, especially matrix-assisted laser desorption/ionization time-of-flight mass spectrometry (MALDI-TOF MS), are amenable to automation (Ross et al. (1997) Anal. Chem. 69:4197-202; Haff et al. (1997) Genome Res. 7:378-88; Ross et al. (2000) Biotechniques 29:620-9; and Roses (2000) Lancent 355:1358-61).

The availability of genome-wide data on DNA variation is thus likely to expand progress in prevention, diagnosis, and treatment customized to the needs of a specific patient, rather that to a statistical average. Further, SNPs provide a new tool for familial linkage and population-based association studies to speed up the identification of genes as targets for new diagnostics and therapeutics (Risch (2000) Nature 405:847-56; and Chakravarti (1999) Nat. Genet. 21: (Suppl. 1):56-60). Moreover, information on DNA variations in human populations can be integrated with an understanding of entire networks of genes and the predication of the contributions of genes in complex disorders based on long-standing interactions between many genes and environmental factors (including lifestyle).

In addition, accurate and high throughput detection of genetic variation and mutation in other species such as viruses is also highly desirable. Viral mutation plays important roles in infection, drug sensitivity, drug resistance, and escaping the immunological response generated to vaccines. For example, Hepatitis B virus (HBV) is such a virus that is notorious for its diverse sero-types and genotypes, as reflected by various mutations in the genome.

Chronic HBV infection poses a serious health threat throughout the world. In the U.S. alone, 5,000 die each year from hepatitis B and its complications; an estimated 1.25 million Americans are chronically infected. Worldwide, more than 400 million people carry the virus. Patients with chronic HBV infection are commonly treated with nucleoside analogs, such as lamivudine (3TC). Lamivudine therapy rapidly decreases serum virus titer, resulting in a profound improvement in clinical symptoms. Unfortunately, prolonged treatment is often associated with the emergence of drug-resistant HBV species. Resistance can take the form of genotypic succession—successive changes of different resistant mutants or a single mutant may dominate. In either case, lamivudine-resistant species result from specific amino acid substitutions in the HBV-encoded polymerase (pol). The most common mutations occur in the YMDD motif, where either valine (codon GTG) or isoleucine (codon ATT) is substituted for methionine (codon ATG).

An increasing number of mutations have also emerged following vaccination. While those mutants that have escaped vaccination are mainly characterized by mutation in the antigenic hepatitis B surface antigen (HBsAg), those carrying mutations in other viral proteins are either resistant to antiviral therapy or implicated in acute liver diseases. Molecular identification of these various mutants should shed new lights on the underlying mechanism of HBV viral escape and drug resistance and provide effectives methods for the diagnosis and treatment of the infection of HBV.

### SUMMARY OF THE INVENTION

The present invention provides methods, compositions and kits for detecting mutation or variation of nucleotide of an organism. Particularly, highly efficient, high throughput assays are developed for detecting mutation or nucleotide variation of an organism by exploiting specific molecular interactions between strands of nucleic acid, and between nucleic acid and protein. More particularly, high throughput, accurate immunoassays are developed for detecting SNP in various organisms including human and human viruses based on specific three-way interactions between Holliday junction (HJ) and its specific binder, and between the HJ-binder and its receptor. This innovative approach circumvents technical and economic problems associated with other SNP detection methods, such as real-time PCR, melting temperature analyses, RFLP, and direct detection of SNP using oligonucleotide arrays.

In one aspect of the invention, a method is provided for detecting nucleotide sequence variation of a target nucleic acid relative to that of a reference nucleic acid.

In one embodiment, the method comprises the steps of: providing a Holliday junction structure formed between a target nucleic acid and a reference nucleic acid, the reference nucleic acid differing in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; and detecting the presence of the Holliday junction structure in the second complex, wherein the presence of the Holliday junction structure in the second complex is indicative of the sequence difference between the target nucleic acid and the reference nucleic acid.

In another embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; and detecting the presence of the Holliday junction structure in the second complex, wherein the presence of the Holliday junction structure in the second complex is indicative of the sequence difference between the target nucleic acid and the reference nucleic acid.

According to the embodiment, the step of detecting the presence of the Holliday junction structure in a second complex includes, but is not limited to, detecting the presence of one or more strands of the Holliday junction by colorimetric detection, fluorescence detection, chemiluminescent detection, enzymatic reaction, gel electrophoresis, mass spectroscopy, or oligonucleotide array.

In another embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder, the receptor being immobilized to a substrate; forming a second complex between the first complex and the receptor for the Holliday junction-binder; and detecting the presence of the Holliday junction structure in the second complex, wherein the presence of the Holliday junction structure in the second complex is indicative of the sequence difference between the target nucleic acid and the reference nucleic acid.

According to the embodiment, the substrate to which the receptor for Holliday junction-binder is immobilized is a solid support such as a microsphere bead, a magnetic bead, a well of a culture plate, glass, membrane or other fabrics.

According to one variation of the embodiment, the method may further comprise the step of isolating the second complex before the step of detecting the presence of the Holliday junction structure in the second complex. The step of isolating includes, but is not limited to, immunoprecipitation, gel electrophoresis, affinity chromatography, oligonucleotide array and flowing fluid sorting.

According to the variation of the embodiment, the step of detecting the presence of the Holliday junction structure in the isolated second complex includes, but is not limited to, detecting the presence of one or more strands of the Holliday junction by colorimetric detection, fluorescence detection, chemiluminescent detection, enzymatic reaction, gel electrophoresis, mass spectroscopy, or oligonucleotide array.

In yet another embodiment, the method comprises the steps of: contacting a target nucleic acid labeled with a tag with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; and detecting the presence of the tag on the target nucleic acid in the Holliday junction structure in the second complex, wherein the presence of the tag on the target nucleic acid in the Holliday junction structure in the second complex is indicative of the sequence difference between the target nucleic acid and the reference nucleic acid.

According to the embodiment, examples of the tag include, but are not limited to, biotin, digoxigenin, fluorescent molecule (e.g., fluorescin and rhodamine), chemiluminescent moiety (e.g., luminol), coenzyme, enzyme substrate, radio isotopes, a particle such as latex or carbon particle, nucleic acid-binding protein, and polynucleotide.

In one variation of the embodiment, the receptor for the Holliday junction-binder is immobilized to a substrate such as a solid support.

According to this variation of the embodiment, the method may further comprise the step of isolating the second complex before the step of detecting the presence of the Holliday junction structure in the second complex. The step of isolating includes, but is not limited to, immunoprecipitation, gel electrophoresis, affinity chromatography, oligonucleotide array and flowing fluid sorting.

In yet another embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; labeling one or more strand of the target or reference nucleic acid in the first complex with a tag; contacting the first complex with a receptor for the Holliday junction-binder that specifically recognizes the Holliday junction-binder; forming a second complex between the first complex and the receptor for the Holliday junction-binder; and detecting the presence of the tag on the Holliday junction structure in the second complex.

In yet another embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; labeling one or more strand of the target or reference nucleic acid in the second complex with a tag; detecting the presence of the tag on the Holliday junction structure in the second complex.

In yet another embodiment, the method comprises the steps of: contacting a target nucleic acid labeled with a tag with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a protein that specifically recognizes a Holliday junction; contacting the first complex with an antibody that specifically binds to the protein that specifically recognizes a Holliday junction; forming a second complex between the first complex and the antibody; and detecting the presence of the tag on the target nucleic acid in the Holliday junction structure in the second complex, wherein the presence of the tag on the target nucleic acid in the Holliday junction structure in the second complex is indicative of the sequence difference between the target nucleic acid and the reference nucleic acid.

According to the embodiment, examples of the tag include, but are not limited to, biotin, digoxigenin, fluorescent molecule (e.g., fluorescin and rhodamine), chemiluminescent moiety (e.g., luminol), coenzyme, enzyme substrate, radio isotopes, a particle such as latex or carbon particle, nucleic acid-binding protein, and polynucleotide.

According to one variation of the embodiment, the antibody that specifically binds to the protein that specifically recognizes a Holliday junction is immobilized to a substrate such as a solid support (e.g., a well of a microplate).

When the tag is biotin, the method may further comprise: contacting the second complex with an agent that comprises streptavidin conjugated to an enzyme such as alkaline phosphatase, perioxidase, or urease.

According to any of the above methods, the target nucleic acid may be derived from a test nucleic acid. The test nucleic acid may be in a purified or unpurified form including DNA (dsDNA and ssDNA) and RNA, including t-RNA, m-RNA, r-RNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and the like.

The target nucleic acid may be a single-stranded or double-stranded nucleic acid. Preferably, the target nucleic acid is double-stranded. The target nucleic acid may be a polynucleotide having a first region that is substantially homologous to the targeted region on the test nucleic acid and a second region that is not necessarily substantially homologous to the targeted region on the test nucleic acid, the second region being designated as a Tail which may be a random or an arbitrary, predetermined sequence.

Optionally, the target nucleic acid comprises a combination of a Target-Tail-1 polynucleotide and a Target-Tail-2 polynucleotide. The Target-Tail-1 polynucleotide and the Target-Tail-2 polynucleotide may differ from each other only in the sequence of Tail-1 and Tail-2. Due to the mismatching sequences of Tail-1 and Tail-2, Target-Tail-1 polynucleotide and the Target-Tail-2 polynucleotide or their respective complementary strands can form a partial duplex which can efficiently initiate spontaneous strand migration when mixed with the reference nucleic acid. The sequence of Tail-1 or Tail-2 may be a random or an arbitrary, predetermined sequence.

Preferably, the target nucleic acid is generated by PCR amplification by using primers targeting a SNP site of the test nucleic acid. More preferably, the reverse primer may further comprise a Tail. Most preferably, two reverse primers comprising Tail-1 and Tail-2, respectively, may be used in the PCR amplification of the test nucleic acid to generate double-stranded Target-Tail-1 polynucleotide and Target-Tail-2 polynucleotide.

According to any of the above methods, the reference nucleic acid may be related to the target nucleic acid in that the two sequences are identical or different from the target nucleic acid in one or more nucleotide positions.

The reference nucleic acid may be DNA, RNA, or PNA that is of sufficient length to form an HJ structure with the target nucleic acid when the reference nucleic acid differs from the target nucleic acid in sequence.

The reference nucleic acid may be a single-stranded or double-stranded nucleic acid. Preferably, the reference nucleic acid is double-stranded. The sequence of the reference nucleic acid may also comprise a Tail.

Optionally, the reference nucleic acid comprises a combination of a Reference-Tail-1 polynucleotide and a Reference-Tail-2 polynucleotide. The Reference-Tail-1 polynucleotide and the Reference-Tail-2 polynucleotide may differ from each other only in the sequence of Tail-1 and Tail-2. Due to the mismatching sequences of Tail-1 and Tail-2, Reference-Tail-1 polynucleotide and the Reference-Tail-2 polynucleotide or their respective complementary strands can form a partial duplex which can efficiently initiate spontaneous strand migration when mixed with the target nucleic acid. The sequence of Tail-1 or Tail-2 may be a random or an arbitrary, predetermined sequence.

Optionally, the reference nucleic acid is generated by PCR amplification by using primers targeting a SNP site of the test nucleic acid. The reverse primer may further comprise a Tail. Also optionally, two reverse primers comprising Tail-1 and Tail-2, respectively, may be used in the PCR amplification of the test nucleic acid to generate double-stranded Reference-Tail-1 polynucleotide and Reference-Tail-2 polynucleotide. Optionally, the reference nucleic acid may be chemically synthesized.

The target or reference nucleic acid sequence usually contains from about 10 to 20,000 or more nucleotides, preferably 30 to 1,000 nucleotides, more preferably 50 to 200 nucleotides, and most preferably 60 to 90 nucleotides.

The reference nucleic acid may be obtained by chemical synthesis or by PCR amplification of a selected nucleic acid template. Preferably, the reference nucleic acid comprises a tail having a random sequence that is not necessary complementary to the test nucleic acid sequence. Most preferably, two reference nucleic acids each having such a tail that differs from each other are used in the formation of the Holliday junction.

In another aspect of the invention, a kit is provided for detecting nucleotide variation between a target nucleic acid and a reference nucleic acid.

In one embodiment, the kit comprises: a reference nucleic acid; forward and reverse target primers for amplifying a targeted region in a test nucleic acid to generate a target nucleic acid; a Holliday junction-binder; and a receptor for the Holliday junction-binder.

According to the embodiment, the kit may further comprise: instructions for how to use the kit to detect mutation or nucleotide variation in a sample containing the test nucleic acid.

Also according to the embodiment, the receptor for the Holliday junction-binder is an antibody that is attached to a substrate such as solid support (e.g., beads, wells of a microplate, resin, etc.).

Also according to the embodiment, when the target nucleic acid or reference nucleic acid is labeled with biotin, the kit may further comprise: streptavidin conjugated to an enzyme.

Also according to the embodiment, the kit may further comprise: internal control primers for amplifying a region different from the targeted region of the test nucleic acid.

In another embodiment, the kit comprises: forward and reverse reference primers for amplifying a targeted region in a test nucleic acid to generate a reference nucleic acid; forward and reverse target primers for amplifying a targeted region in a test nucleic acid to generate a target nucleic acid; a Holliday junction-binder; and a receptor for the Holliday junction-binder.

According to any of the above embodiment, the Holliday junction-binder is preferably a protein that specifically binds a stabilized Holliday junction. Examples of such protein include, but are not limited to, resolvases and recombinases such as RuvA, RuvC, RuvB, RusA, RuvG of E. coli; proteins/mutants derived from RuvA, RuvC, RuvB, RusA, RuvG, homologs (including functional homologs) of RuvA, RuvC, RuvB, RusA, RuvG from various other organisms, such as the homologs of RuvA, RuvC, RuvB, RusA, and RuvG derived from mammals, Cce1 and spCce1 from yeast, Hjc from Pyrococcus furiosusa; thermostable proteins such as thermostable homologs of RuvA, RuvC, RuvB, RusA, and RuvG that are derived from thermophilic organisms—organisms selected from the group consisting of Thermus aquaticus, Thermus flavus, and Thermus thermophilus.

Also optionally, the HJ-binder may be a recombinant resolvase or recombinase that is conjugated or fused with a moiety (e.g., a His-tag) can be used for specific binding to stabilized Holliday junction so long as the moiety does not substantially interfere with the specific binding interaction.

According to any of the above embodiments, the receptor for a Holliday junction-binder is preferably an antibody that specifically binds to the Holliday junction. The antibody may be a monoclonal, polyclonal, Fab, fragments of the variable regions, single-chain antibody, or antibody contained in anti-serum.

The above-described methods, compositions and kits may be used in a wide variety of applications such as diagnostics, genotyping, genetic profiling, mutation detection, disease prevention, therapeutic treatment, and screening for therapeutic targets or therapeutics.

### BRIEF DESCRIPTION OF THE FIGURES

FIG. 1 is a flow chart illustrating an embodiment of a Holliday junction-based assay for detecting mutation or nucleotide variation in an organism.

FIG. 2 is a flow chart illustrating a particular embodiment of a Holliday junction-based immunoassay for detecting mutation or nucleotide variation in an organism.

FIG. 3 is a flow chart outlining the formation and detection of allele-specific Holliday junction by PCR amplification and branch migration inhibition.

FIG. 4 shows typical primer design for amplification of the target region and reference DNA by PCR.

FIG. 5 shows that a PAGE-based Holliday junction allele-specific genotyping method was used to genotype the HFE C282Y mutation on 80 genomic DNA samples.

FIG. 6 shows results of Holliday junction structures analyzed on PAGE.

FIG. 7 shows effects of RuvA concentration on the HJ binding tested by a gel shift assay.

FIG. 8 illustrate an ELISA for detecting nucleotide difference according to the present invention.

FIG. 9 shows results of the optimization of anti-RuvA antibody concentration and sample amounts on human Factor V genotyping system.

FIG. 10 shows the effect of different branch migration temperatures on the ELISA sensitivity at lower RuvA concentrations.

FIG. 11 shows the effect of binding buffer on background level in the ELISA.

FIG. 12 shows results of genotyping of samples.

FIG. 13 shows results of detection of mutation in human Factor II and MTHFR mutation using methods of the present invention.

FIG. 14 shows the effect of HRP concentration on efficiency of detection in such a single-wash ELISA as compared to that in a standard protocol involving 2 separate steps of antibody binding and HRP labeling.

FIG. 15 shows a purification profile of C-terminal 6×His-tagged RuvA protein using a Ni-NTA column.

FIG. 16 shows that C-terminal 6×His-tagged RuvA protein is fully functional under the condition of Holliday-junction based ELISA for genotyping (Panel A: comparison with that of no His-tagged recombinant RuvA using a gel shift assay; and Panel B: the His-tagged RuvA protein tested in the Holliday junction-based ELISA for genotyping).

FIG. 17 shows primers designed for testing mutations in the YMDD motif of HBV.

FIG. 18 shows results obtained from PAGE experiments on detecting two different point mutations occur at same codon in the HBV polymerase gene based on formation of Holliday junction.

FIG. 19 shows averaged results of detection of mutation in HBV YMDD motif by using a two-step PCR/branch migration protocol and two-wash ELISA protocol.

### DETAILED DESCRIPTION OF THE INVENTION

The present invention provides methods, compositions and kits for highly efficient, high throughput detection of mutation or nucleotide variation of an organism. By exploiting the molecular interactions between strands of nucleic acid and between nucleic acid and protein, assays have been developed to detect nucleotide variation, in particular, single nucleotide polymorphism in various biological samples. In preferred embodiments, immunoassays are developed to specifically capture a nucleic acid-protein complex formed between a 4-way nucleic acid structure called Holliday junction and a protein that specifically recognizes the Holliday junction. These assays can be used in a wide variety of applications such as diagnostics, genotyping, genetic profiling, mutation detection, disease prevention, therapeutic treatment, and screening for therapeutic targets or therapeutics.

1. Method for Detection of Nucleotide Variation

Most of the genetic differences between individuals can be attributed to single nucleotide polymorphisms, or SNPs. The frequency of SNPs in the human genome is approximately one per 1,000 base pairs. Although most occur in noncoding regions of the genome, SNPs within genes can produce devastating effects. Many different diseases—from heart disease to cystic fibrosis—have been attributed to specific SNPs, and the list continues to grow.

Although several different genotyping technologies are currently used to detect disease-causing SNPs, none of these methods is ideal. Real-time PCR and melting temperature analyses have the advantages of minimal manual manipulation, easy automation, and high-throughput capability; however, these methods are prohibitively expensive for many researchers. In contrast, comparatively inexpensive techniques such as RFLP and site-specific primer PCR are labor intensive, time consuming, and low throughput. Single-base extension, another popular technology, is inflexible and requires extensive sample manipulation.

The present invention provides an innovative approach to circumventing these problems. Specifically, highly efficient and sensitive assays are developed to detect the presence of nucleotide variation in genomic DNA, especially SNPs. In particular, these assays are based on the capture and detection of branch structures (e.g., Holliday junction) formed between target DNA and reference DNA strands. The capture of the Holliday junction (HJ) may be accomplished by specific binding of the HJ to an HJ-binder, such as a HJ-binding protein, resolvase. These assays are readily adaptable for high throughput, automated detection of SNPs in multiple samples.

In one aspect of the invention, a method is provided for detecting nucleotide sequence variation of a target nucleic acid relative to that of a reference nucleic acid.

In one embodiment, the method comprises the steps of: providing a Holliday junction structure formed between a target nucleic acid and a reference nucleic acid, the reference nucleic acid differing in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; and detecting the presence of the Holliday junction structure in the second complex, wherein the presence of the Holliday junction structure in the second complex is indicative of the sequence difference between the target nucleic acid and the reference nucleic acid.

In another embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; and detecting the presence of the Holliday junction structure in the second complex, wherein the presence of the Holliday junction structure in the second complex is indicative of the sequence difference between the target nucleic acid and the reference nucleic acid.

According to the embodiment, the step of detecting the presence of the Holliday junction structure in a second complex includes, but is not limited to, detecting the presence of one or more strands of the Holliday junction by colorimetric detection, fluorescence detection, chemiluminescent detection, enzymatic reaction, gel electrophoresis, mass spectroscopy, or oligonucleotide array.

In another embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder, the receptor being immobilized to a substrate; forming a second complex between the first complex and the receptor for the Holliday junction-binder; detecting the presence of the Holliday junction structure in the second complex, wherein the presence of the Holliday junction structure in the second complex is indicative of the sequence difference between the target nucleic acid and the reference nucleic acid.

According to the embodiment, the substrate to which the receptor for Holliday junction-binder is immobilized is a solid support such as microsphere bead, magnetic bead, a well of a culture plate, glass, membrane or other fabrics.

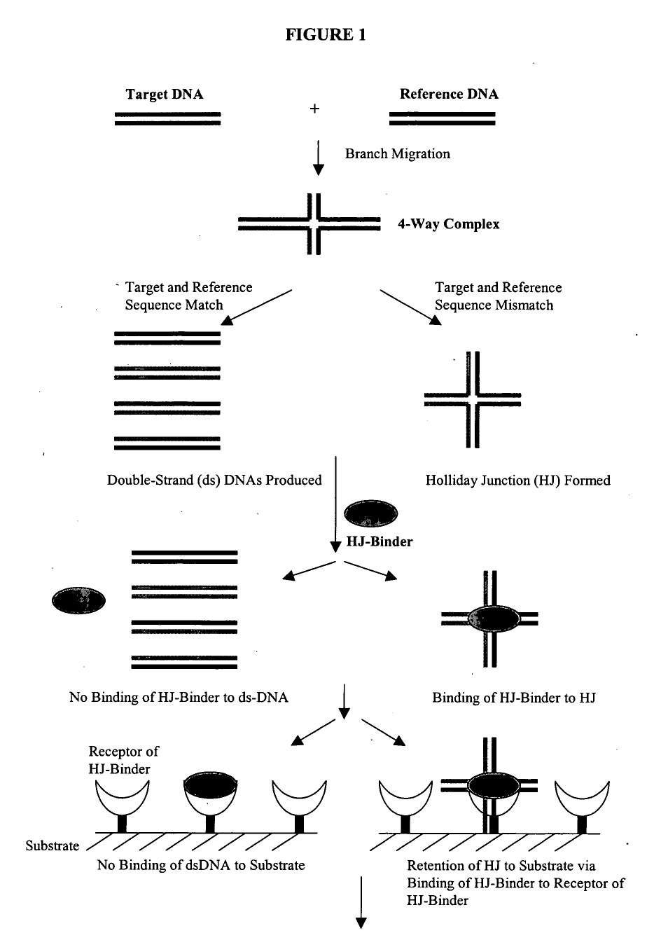

FIG. 1 schematically illustrates an example of such an assay for capturing and detecting the presence of an HJ structure. As illustrated in FIG. 1, target nucleic acid strands such as double-stranded DNA can be prepared, for example, by amplifying genomic DNA using designed primers to target a specific region such as one that contains or is suspected of containing SNP. The double stranded amplicon can be mixed with reference DNA strands that are designed to match or mismatch the targeted region of the genomic DNA. The mixture is subjected to branch migration condition (e.g., 60-65° C. for 30 min) such that a 4-way complex is formed. If the target DNA strands and reference DNA strand match each other in sequence, double-stranded (ds) DNA will form upon completion of the branch migration process. However, if the target DNA strands and reference DNA strand do not match each other in sequence, i.e., mismatch, a stabilized HJ structure will form upon completion of the branch migration process.

Referring to FIG. 1, with addition of an HJ-binding protein to the branch migration products, the HJ structure will bind specifically to the HJ-binder and form a DNA-protein complex. Such a DNA-protein complex can be captured by using a receptor that specifically recognizes the HJ-binder protein. If the receptor for the HJ-binding protein is immobilized on a solid substrate, the HJ structure can then be captured and immobilized on the substrate as a result. The presence of the HJ structure on the substrate can then be detected using various methods.

The various components that may be needed for the assays of the present invention are described in detail as follows.

1) Target Nucleic Acid

In the present invention, a target nucleic acid refers to a sequence of nucleotides to be studied either for the presence of a difference from a reference sequence or for the determination of its presence or absence. The target nucleic acid sequence may be double stranded or single stranded and from a natural or synthetic source. When the target nucleic acid sequence is single stranded, a nucleic acid duplex comprising the single stranded target nucleic acid sequence may be produced by primer-extension and/or amplification.

The target nucleic acid includes both nucleic acid and fragments thereof from any source, in purified or unpurified form including DNA (dsDNA and ssDNA) and RNA, including t-RNA, m-RNA, r-RNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and the like. For example, the target nucleic acid may be collected from an individual to be tested for SNP in a particular region or regions of his/her genome, or to be tested for mutation of viral strain harbored in his/her body.

The target nucleic acid can be only a minor fraction of a complex mixture such as a biological sample. Examples of a biological sample include, but are not limited to, biological fluids such blood, serum, plasma, sputum, lymphatic fluid, semen, vaginal mucus, feces, urine, spinal fluid, and the like; biological tissue such as hair and skin; cell cultures; plants; food; forensic samples such as paper, fabrics and scrapings; water; sewage; and medicinals.

The target nucleic acid can also be an unnatural, synthetic polymer such as peptide-nucleic acid (PNA) that is composed of peptide backbone but retains base residues of a DNA or RNA.

The target nucleic acid sequence usually contains from about 10 to 20,000 or more nucleotides, preferably 30 to 1,000 nucleotides, more preferably 50 to 200 nucleotides, and most preferably 60 to 90 nucleotides. The target nucleic acid sequence is generally a fraction of a larger molecule or it may be substantially the entire molecule. The minimum number of nucleotides in the target sequence is selected to assure that a determination of a difference between the target nucleic acid and the reference nucleic acid in accordance with the present invention can be achieved.

The target nucleic acid may be a single-stranded or double-stranded nucleic acid. Preferably, the target nucleic acid is double-stranded. The target nucleic acid may be a polynucleotide having a first region that is substantially homologous (preferably, at least 80% homologous, more preferably at least 90% homologous, and most preferably at least 95% homologous) to the targeted region on the test nucleic acid and a second region that is not necessarily substantially homologous to the targeted region on the test nucleic acid, the second region being designated as a Tail which may be a random or an arbitrary, predetermined sequence.

Optionally, the target nucleic acid comprises a combination of a Target-Tail-1 polynucleotide and a Target-Tail-2 polynucleotide. The Target-Tail-1 polynucleotide and the Target-Tail-2 polynucleotide may differ from each other only in the sequence of Tail-1 and Tail-2. Due to the mismatching sequences of Tail-1 and Tail-2, Target-Tail-1 polynucleotide and the Target-Tail-2 polynucleotide or their respective complementary strands can form a partial duplex which can efficiently initiate spontaneous strand migration when mixed with the reference nucleic acid. The sequence of Tail-1 or Tail-2 may be a random or an arbitrary, predetermined sequence.

Preferably, the target nucleic acid is generated by PCR amplification by using primers targeting a SNP site of the test nucleic acid. More preferably, the reverse primer may further comprise a Tail. Most preferably, two reverse primers comprising Tail-1 and Tail-2, respectively, may be used in the PCR amplification of the test nucleic acid to generate double-stranded amplicons: Target-Tail-1 polynucleotide and Target-Tail-2 polynucleotide.

2) Reference Nucleic Acid

In the present invention, a reference nucleic acid refers to a nucleic acid sequence that is related to the target nucleic acid in that the two sequences are identical or different from the target nucleic acid in one or more nucleotide positions.

The reference nucleic acid may be DNA, RNA, or PNA that is of sufficient length to form an HJ structure with the target nucleic acid when the reference nucleic acid differs from the target nucleic acid in sequence.

The reference nucleic acid sequence usually contains from about 10 to 20,000 or more nucleotides, preferably 30 to 1,000 nucleotides, more preferably 50 to 200 nucleotides, and most preferably 60 to 90 nucleotides.

The reference nucleic acid may be DNA, RNA, or PNA that is of sufficient length to form an HJ structure with the target nucleic acid when the reference nucleic acid differs from the target nucleic acid in sequence.

The reference nucleic acid may be a single-stranded or double-stranded nucleic acid. Preferably, the reference nucleic acid is double-stranded. The sequence of the reference nucleic acid may also comprise a Tail.

Optionally, the reference nucleic acid comprises a combination of a Reference-Tail-1 polynucleotide and a Reference-Tail-2 polynucleotide. The Reference-Tail-1 polynucleotide and the Reference-Tail-2 polynucleotide may differ from each other only in the sequence of Tail-1 and Tail-2. Due to the mismatching sequences of Tail-1 and Tail-2, Reference-Tail-1 polynucleotide and the Reference-Tail-2 polynucleotide or their respective complementary strands can form a partial duplex which can efficiently initiate spontaneous strand migration when mixed with the target nucleic acid. The sequence of Tail-1 or Tail-2 may be a random or an arbitrary, predetermined sequence.

Optionally, The reference nucleic acid may be a “reference amplicon” from a sample containing the test nucleic acid. Primers may be designed to incorporate the same targeted nucleotide(s) or different nucleiotide(s) such as the common variant or mutant of the wildtype genome. Preferably, the reference nucleic acid is generated by PCR amplification by using primers targeting a SNP site of the test nucleic acid. The reverse primer may further comprise a Tail. Also optionally, two reverse primers comprising Tail-1 and Tail-2, respectively, may be used in the PCR amplification of the test nucleic acid to generate double-stranded amplicons: Reference-Tail-1 polynucleotide and Reference-Tail-2 polynucleotide. Optionally, the reference nucleic acid may be chemically synthesized.

3) Tag for Target or Reference Nucleic Acid

To facilitate detection of target or reference nucleic acid, one or more tag may be added to the target or reference nucleic acid using various methods for labeling nucleic acid. The tag may be covalently conjugated with the nucleic acid or non-covalently attached to the nucleic through sequence-specific or non-sequence-specific binding.

Examples of the tag includes, but are not limited to biotin, digoxigenin, fluorescent molecule (e.g., fluorescin and rhodamine), chemiluminescent moiety (e.g., luminol), coenzyme, enzyme substrate, radio isotopes, a particle such as latex or carbon particle, nucleic acid-binding protein, polynucleotide that specifically hybridizes with either the target or reference nucleic acid strand. Detection of the presence of the tag can be achieved by observation or measurement of signals emitted from the tag. The production of the signal may be facilitated by binding of the tag to its counter-part molecule, which triggers a reaction directly or indirectly. For example, the target or reference nucleic acid may be both labeled with biotin, upon binding of streptavidin-HRP (horse radish peroxidase) and addition of the substrate for HRP (e.g., ABTS), the presence of the biotin-labeled HJ can be detected by observing or measuring color changes in the mixture.

4) Branch Migration and Formation of Holliday Junction (HJ)

During genetic recombination, two duplexes of DNA partners exchange DNA strands, an intermediate of which is a 4-way branch structure termed “Holliday junction” (Holliday (1964) Genet. Res. Camb. 5:282-304). The Holliday junction is capable of undergoing branch migration resulting in dissociation into two double stranded sequences where sequence identity and complementarity extend to the ends of the strands. Under suitable branch migration conditions branch migration will proceed only if strand exchange does not result in a mismatch, wherein the formation of a single base mismatch will impede branch migration, resulting in a stabilized Holliday junction or Holliday junction complex. Appropriate conditions can be found, for example, in Panyutin and Hsieh, (1993) J. Mol. Biol., 230:413-24. In certain applications the conditions will have to be modified due to the nature of the particular polynucleotides involved. Such modifications are readily discernible by one of skill in the art without undue experimentation.

Preferably, double-stranded target nucleic acid is contacted with double-stranded reference DNA before the initiation of branch migration. If the target nucleic acid or the reference nucleic acid is single-stranded, denaturing of the mixture and/or annealing of the target and reference strands may be conducted before the initiation of branch migration.

5) HJ-Binders

According to the present invention, an agent that specifically binds to HJ structure is used to capture HJ formed between the target and reference nucleic acid. In a preferred embodiment, a protein or proteins is used to bind a stabilized Holliday junction. Many proteins from various organisms have been shown specifically bind to Holliday junctions. Those proteins include but are not limited to resolvases and recombinases such as RuvA, RuvC, RuvB, RusA, RuvG of E. coli; proteins/mutants derived from RuvA, RuvC, RuvB, RusA, RuvG. In addition, such proteins include homologs (including functional homologs) of RuvA, RuvC, RuvB, RusA, RuvG from various other organisms, such as the homologs of RuvA, RuvC, RuvB, RusA, and RuvG derived from mammals, Cce1 and spCce1 from yeast, Hjc from Pyrococcus furiosusa, and various other resolvases and recombinases that can specifically bind to Holliday structures.

Optionally, thermostable proteins are used to bind to a stabilized Holliday structure. Such thermostable proteins include thermostable homologs of RuvA, RuvC, RuvB, RusA, and RuvG that are derived from thermophilic organisms—organisms selected from the group consisting of Thermus aquaticus, Thermus flavus, Thermus thermophilus and other thermophilic organisms known to those of skill in the art. Hjc from Pyrococcus furiosusa is one good example of an appropriate thermostable protein with specificity for Holliday structures.

Also optionally, a recombinant resolvase or recombinase that is conjugated or fused with a moiety (e.g., a His-tag) can be used for specific binding to stabilized Holliday junction so long as the moiety does not substantially interfere with the specific binding interaction.

The preparation and properties of a number of such proteins useful in the practice of the present invention have been described, for example, in the following list of literature references, all of which are incorporated herein in their entirety: Davies and West, supra; Whitby et al., supra; Iwasaki H,. et al. 1992. E. coli RuvA and RuvC proteins specifically interact with Holliday Junctions and promote branch migration. Genes Dev. 6:2214-20; Parsons Calif., et al. 1992. Interaction of E. Coli RuvA and RuvB proteins with synthetic Holliday junctions. Proc. Natl. Acad. USA 89:5452-56; Traneva IR, et al. 1992. Purification and properties of the RuvA and, RuvB proteins of E. coli. Mol. Gen. Genet. 235:1-10; Rafferty J B, et al. 1996. Crystal structure of the DNA recombination protein RuvA and a model for its binding to the Holliday junction. Science 274:415-21; Hargreaves D., et al. 1999. Crystalization of E. coli RuvA complexed with a synthetic Holliday junction. Acta Crystallogr D. Biol Crystallogr 55(Pt 1):263-5; Hargreaves D., et al. 1998. Crystal structure of E. coli RuvA with bound DNA Holliday junction at 6A resolution. Nature Struct Biol. 5(6):441-6; Dunderdale H J, et al. 1994. Cloning, overexpression, purification, and characterization of the E. coli RuvC Holliday junction resolvase. J Biol Chem 267 (7):5187-94; Ariyoshi M, et al. 1994. Atomic structure of the RuvC resolvase: a Holliday junction specific endo nuclease from E. coli. Cell 78(6): 1063-72; Sharples G J, et al. 1994. Processing of intermediates in recombination and DNA repair: identification of a new endonuclease that specifically cleaves Holliday junction. EMBO 13(24):6133-42; Rice P, et al. 1995. Structure of the bacteriophage Mu transposase core: a common structural motif for DNA transposition and retroviral integration. Cell 82(2):209-20; Bujacz G., et al. 1995. High-resolution structure of the catalytic domain of avian sarcoma virus integrase. J Mol Biol 253(2):333-46; Rice P. et al. 1996. Retroviral integrases and their cousins. Curr Opin Struct Biol 6(1):76-83; Suck D. 1997. DNA recognition by structure-selective nucleases. Biopolymer 44(4):405-21; White M F, et al. 1997. The resolving enzyme CCE1 of yeast opens the structure of the four-way junction. J Mol Biol 266(1):122-34; Whitby M C, et al. 1997. A new Holliday junction resolving enzyme from S. pombe that is homologous to CCE1 from S. cerevisiae. J Mol Biol 271(4):509-22; Bidnenko E, et al. 1998. Lactococcus lactis phage operon coding for an endonuclease homologous to RuvC. Mol Microbiol 28(4): 823-34; Raaijmakers H, et al. 1999. X-ray structure of T4 endonuclease VII: a DNA junction resolvase with a novel fold and unusual domain-swapped dimer achitecture. EMBO. 18(6):1447-58; Komori K, et al. 1999. A Holliday junction resolvase from Pyrococcus furiosus: functional similarity to E. coli RuvC provides evidence for conserved mechanism of homologous recombination in Bacteria, Eukarya, and Archaea. Proc Natl Acad Sci USA. 96(16):8873-8; Komori K, et al. 2000. Mutational analysis of the Pyrococcus furiosus Holliday junction resolvase Hjc revealed functionally important residues for dimer formation, junction DNA binding and cleavage activities. J. Biol. Chem. (September/2000 issue); Sharples G J, et al. 1999. Holliday junction processing in bacteria: insight from the evolutionary conservation of RuvABC, RecG, and RusA. J bacteriol 181(8);5543-50; Sharples G J, et al. 1993. An E. coli RuvC mutant defective in cleavage of synthetic Holliday junctions. Nucleic Acid Research, 21(15): 3359-64.

6) Isolation of the Complex Formed Between HJ and HJ-Binder

According to the present invention, the stabilized Holliday junction may be isolated from the branch migration mixture based on specific interaction between the HJ and an HJ-binder. Through specific binding between the HJ and the HJ-binder, other components in the mixture, such as duplexes and single stranded polynucleotides, can be separated from the HJ-HJ-binder complex.

There are several ways to separate the complex from the branch migration mixture, such as immunoprecipitation, gel electrophoresis, capillary electrophoresis, and affinity chromatography. Preferably, immunoprecipitation is used to separate HJ from the branch migration mixture.

Typically, immunoprecipitation involves the interaction between a protein and its specific antibody, the separation of these immune complexes with Protein G or Protein A, and the subsequent analysis by SDS-PAGE. Alternatively, the antibody-protein complex is precipitated from the solution using an insoluble resin that binds to the antibody complex (such as Protein A or Protein G immobilized on a solid support, Staphlococcus aureus cells and affinity resin with covalently attached secondary antibody). Unbound proteins and other molecules are removed by washing the resin. This technique provides a rapid and simple means to separate HJ-HJ-binder complex from duplexes and single stranded polynucleotides in the branch migration mixture.

Preferably the HJ-binder is a resolvase such as RuvA, RuvC, RuvB, RusA, RuvG of E. coli and functional equivalent protein thereof. Antibody against the resolvase such as RuvA may be used as the primary antibody in immunoprecipitation.

The success of immunoprecipitation may depend on the affinity of the antibody for its antigen as well as for Protein G or Protein A. In general, while polyclonal antibodies are best, purified monoclonal antibodies (mAb), ascites fluid, or hybridoma supernatant can also be used. The strength of interaction between the mAb and Protein G or Protein A is an important factor in the decision of which slurry to use. Protein G coupled to some insoluble matrix (e.g. sepharose beads) binds well to most subclasses of rat immunoglobulins and mouse IgG1, while Protein A binds much better to mouse IgG2a, IgG2b, and IgG3.

Optionally, affinity chromatography may be used to separate HJ-HJ-binder complex from the rest of the components in branch migration mixture. Affinity chromatography involves using a protein specific for binding to the target analyte, such as an antibody specific for the antigen, to pull the analyte out of a complex mixture. The antibody may be covalently attached to a solid support resin. The branch migration mixture is passed over the attached antibody-affinity resin. While the target antigen binds to the antibody, other components in the mixture simply pass through the column. Once the unbound components are washed off the column, a specific eluant (such as a buffer containing an epitope peptide recognized by the attached antibody) can release the bound antigen from the column. When applied to separation of HJ-HJ-binder complex from the branch migration mixture, the affinity column preferably includes antibody against the HJ-binder, such as antibody against resolvase such as RuvA, RuvC, RuvB, RusA, RuvG of E. coli and functional equivalent protein thereof.

Also preferably, the HJ-HJ-binder complex may be isolated from the branch migration mixture by using a plate precoated with protein specifically binding to the HJ-binder, such as antibody against RuvA. By using a multi-well microplate, the isolation can be performed efficiently and in an automated, high throughput manner.

7) Detection of HJ in the Complex Formed Between HJ and HJ-Binder

The strands of nucleic acid in the HJ present in the complex formed between HJ and HJ-binder may be detected using various methods.

a) Modified ELISA

In a preferred embodiment, the complex formed between HJ and HJ-binder is isolated and detected using a modified ELISA (Enzyme-Linked Immunosorbent Assay). Typically, an ELISA involves the following steps:
i) Bind the sample being tested for the presence of a specific molecule or organism to a solid support such as a plastic microtiter plate, which usually contains 96 sample wells;
ii) Add a merker-specific antibody (primary antibody) to the bound material, and then wash the support to remove unbound primary antibody;
iii) Add a second antibody (secondary antibody) binds specifically to the primary antibody and not to the target molecule. Bound (conjugated) to the secondary antibody is an enzyme such as alkaline phosphatase, perioxidase, or urease, which can catalyze a reaction that converts a colorless substrate into a colored product. Wash the mixture to remove any unbound secondary antibody-enzyme conjugate.
iv) Add the colorless substrate.
v) Observe or measure the amount of colored product.

According to a particular embodiment of the present invention, a modified ELISA is provided for detecting the presence of HJ in the HJ-HJ-binder complex. Antibody that specifically binds to the HJ binder such as a resolvase is used to coat the surface of a plate, preferably a multi-well microplate. The antibody may be a monoclonal, polyclonal, Fab, fragments of the variable regions, single-chain antibody, or antibody contained in anti-serum. Methods for coating an ELISA plate are well known in the art by following standard protocols provided by manufacturers. For example, a 96-well plate can be coated by following this protocol:
Add 100 ul of 1 ug/ml Anti-RuvA monoclonal antibody (in PBS buffer) to each well of 96-well ELISA plate;
   Incubate the plate at room temperature over night;
   Remove the antibody solution completely;
   Wash wells with 400 ul TBS;
   Add 200 ul BSA-TBS blocking buffer;
   Incubate at room temperature for 2 hours;
   Remove the blocking buffer completely;
   Add 200 ul TBS and store at 4° C.

As described above, the HJ-HJ-binder complex, such as HJ-RuvA complex, can be isolated from the branch migration mixture using different method. In this modified ELISA, the HJ-RuvA complex is isolated from the branch migration mixture through specific binding to anti-RuvA antibody coated on the wells of a microplate. To detect the presence of the HJ in the HJ-RuvA complex bound to the substrate of the plate, one or more strands of the HJ may be labeled with a tag either before or after the formation of the Holliday junction.

In one embodiment, one or more strands of the target nucleic acid and/or one or more strands of the reference nucleic acid may be labeled with a tag such as biotin before the formation of the Holliday junction. Upon binding of the HJ-HJ-binder complex to the substrate of the plate, an agent (e.g., streptavidin-HRP) that includes a moiety that specifically recognizes the tag (e.g., biotin) and is conjugated to an enzyme (e.g., HRP) can be added to the wells of the plate. Addition of the substrate for the enzyme would trigger a reaction resulting in color change in the plate.

In another embodiment, one or more strands of the target nucleic acid and/or one or more strands of the reference nucleic acid may be labeled with a tag such as biotin or digoxigenin after the formation of the Holliday junction. For example, one or more strands of the HJ in the HJ-HJ-binder complex may be labeled with biotin or digoxigenin. In this method, terminal transferase is used to add a single modified dideoxyuriding-triphosphate (ddUTP) conjugated with biotin or digoxigenin to the 3′-end of the oligonucleotide after the formation of Holliday junction. Optionally, a biotinylated polyA tail may be added to the 3′-end of the strand by using terminal transferase. Upon binding of the HJ-HJ-binder complex to the substrate of the plate, an agent (e.g., streptavidin-HRP) that includes a moiety that specifically recognizes the tag (e.g., biotin) and is conjugated to an enzyme (e.g., HRP) can be added to the wells of the plate. Addition of the substrate for the enzyme would trigger a reaction resulting in color change in the plate.

b) Fluorescence-Based Detection

In another embodiment, the presence of the HJ in the complex formed between HJ and the HJ-binder may be detected through a fluorescent tag on one or more strands of the HJ.

In one variation of the embodiment, for the above-described ELISA, the tag on the HJ-HJ-binder complex can be replaced with a fluorescent tag. Instead of the enzyme-linked detection, the complex bound to the substrate of a plate can be directly measured by fluorescence emitted from the tag using a fluorescence polarization machine. Fluorescent dyes with diverse spectral properties (e.g., as supplied by Molecular Probes, Eugene, Oreg.) may be used to simultaneously detect multiple SNPs. In this assay, the reference nucleic acid targeting each of the SNPs may be labeled with a fluorescent dye having different spectral property than that for another SNP.

In another variation of the embodiment, different SNPs can be simultaneously detected in an even higher throughput than an assay conducted using microbeads labeled with different spectral property and/or fluorescent (or colorimetric) intensity. For example, polystyrene microspheres are provided by Luminex Corp, Austin, Tex. that are internally dyed with two spectrally distinct fluorochromes. Using precise ratios of these fluorochromes, a large number of different fluorescent bead sets (e.g., 100 sets) can be produced. Each set of the beads can be distinguished by its spectral address, a combination of which allows for measurement of a large number of analytes in a single reaction vessel. A third fluorochrome coupled to a reporter molecule quantifies the biomolecular interaction that has occurred at the microsphere surface. These different fluorescent bead sets can be used to label the reference nucleic acids targeting different SNP sites on a test DNA by using standard nucleic acid chemical synthesis methods or by PCR amplification using primers labeled with the beads (e.g., primers modified with 5′ amine for coupling to carboxylated microsphere or bead).

The fluorescent bead-labeled reference nucleic acid can be used to form HJ with the target DNA containing the SNP site. Because each of the different reference nucleic acid is uniquely labeled with beads with distinguishable spectral address, the resulting HJ will be distinguishable for each different SNP site. After the HJ-HJ-binder complex is isolated, the different SNP sites targeted can be detected by passing the beads through rapidly flowing fluid stream. In the stream, the beads are interrogated individually as they pass two separate lasers. High speed digital signal processing classifies the beads based on its its spectral address and quantifies the reaction on the surface. Thousands of beads can interrogated per second, resulting a high speed, high throughput and accurate detection of multiple different SNPs in samples.

c) Detection Based on Nucleic Acid Hybridization Array

In one embodiment, nucleic acid hybridization can be used for the resolution of the identity of the reference or target nucleic acid in the HJ-HJ-binder complex.

In a variation of the embodiment, nucleic acid hybridization probes may be employed to hybridize to the reference or target nucleic acid present in the HJ-HJ-binder complex, preferably in isolated HJ-HJ-binder complex. The nucleic acid hybridization probes can be designed to contain a detectable marker to facilitate the detection after the hybridization, such as biotin and fluorescence dyes, and/or immobilized to a solid support such as a glass substrate or cellulose membrane.

In a preferred variation of the embodiment, the reference or target nucleic acid present in the HJ-HJ-binder complex can detected through hybridization with a nucleic acid array. For example, an array of hybridization probes designed to target the reference nucleic acid in the HJ-HJ-binder complex can be attached to a solid support where different hybridization probes are attached to discrete, different regions of the array. Each different region of the array comprises one or more copies of a same hybridization probe which incorporates a sequence that is complementary to a partial or full sequence of the reference nucleic acid for each targeted SNP. As a result, the hybridization probes in a given region of the array can selectively hybridize to and immobilize a different reference nucleic acid present in the HJ-HJ-binder complex.

By detecting which regions the isolated reference nucleic acid hybridize to on the array, one can determine which SNP or nucleic acid variation/mutation are present in the sample and can also quantify the amount of SNP or nucleic acid variation/mutation, such as allele frequency.

Numerous methods have been developed for attaching hybridization probes to solid supports in order to perform immobilized hybridization assays and detect target oligonucleotides in a sample. Numerous methods and devices are also known in the art for detecting the hybridization of a target oligonucleotide to a hybridization probe immobilized in a region of the array. Examples of such methods and device for forming arrays and detecting hybridization include, but are not limited to those described in U.S. Pat. Nos. 6,197,506, 6,045,996, 6,040,138, 5,424,186, 5,384,261, each of which are incorporated herein by reference.

Typically, the use of DNA probe arrays to obtain genetic information involves the following general steps: design and manufacture of DNA probe array wafers, preparation of the sample, hybridization of target DNA to the array, detection of hybridization events and data analysis to determine sequence. Preferred wafers are manufactured using a process adapted from semiconductor manufacturing to achieve cost effectiveness and high quality, and are available from Affymetrix, Inc. of California.

Probe arrays can be manufactured by a light-directed chemical synthesis process, which combines solid-phase chemical synthesis with photolithographic fabrication techniques as employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. This parallel process enhances reproducibility and helps achieve economies of scale.

Once fabricated, DNA probe arrays can be used to obtain genetic information about nucleic acid samples. The nucleic acid samples are tagged with a fluorescent reporter group by standard biochemical methods. The labeled samples are incubated with a wafer, and segments of the samples bind, or hybridize, with complementary sequences on the wafer. The wafer is then scanned and the patterns of hybridization are detected by emission of light from the fluorescent reporter groups. Because the identity and position of each probe on the wafer is known, the nature of the nucleic acid sequences in the sample applied to the wafer can be determined. When these arrays are used for genotyping experiments, they may be referred to as genotyping arrays.

Once fabricated the arrays are ready for hybridization. The nucleic acid sample to be analyzed is isolated, amplified and labeled with a fluorescent reporter group. The labeled nucleic acid sample is then incubated with the array using a fluidics station and hybridization oven. After the hybridization reaction is complete, the array is inserted into the scanner, where patterns of hybridization are detected. The hybridization data are collected as light emitted from the fluorescent reporter groups already incorporated into the labeled nucleic acid, which is now bound to the probe array. Probes that most clearly match the labeled nucleic acid bind more of the nucleic acid, and hence accumulate more of the fluorescent signal than those that have mismatches. Since the sequence and position of each probe on the array are known, by complementarity, the identity of the nucleic acid sample applied to the probe array can be identified.

Several modifications may be made to the hybridization arrays known in the art in order to customize the hybridization arrays for use in detecting SNP or nucleic acid variation/mutation through the characterization of isolated HJ that forms a complex with an HJ-binder such as RuvA.

A variety of different libraries of nucleic acid probes can be designed depending on the number of targeted SNP or nucleic acid variation/mutation and the nature and purpose of the investigation. Selection of the sequences used in the hybridization probes may be based on the different SNPs or nucleic acid variation/mutation that one wishes to detect in a sample. This, in turn, may depend on the type of organism, cell, or disease state one wishes to identify SNP or nucleic acid variation/mutation.

By using a hybridization array, multiple different SNP sites or nucleic acid variation/mutation can be detected simultaneously and efficiently. For example, a library of different reference nucleic acids can be mixed with a library of target nucleic acid. After branch migration, different HJ structures form in the mixture. An HJ-binder, such as RuvA, can be added to the branch migration mixture. The HJ-binder is preferably not to be sequence-specific such that the HJ-binder will bind to all of the different HJ structures. As described in detailed above, the HJ-HJ-binder complex may be isolated using various methods. Preferably, the HJ-binder is a resolvase such as RuvA and the receptor for the HJ-binder is an anti-resolvase antibody such as anti-RuvA antibody. The antibody can be used to isolate the HJ-HJ-binder complex by forming an even larger complex involving these three molecules in an assay, such as immunoprecipitation. The nucleic acid strands in the HJ present in the isolated large complex can be analyzed by using a nucleic acid hybrization array.

A positive hybridization signal for a specific SNP on the hybridization array means that the test sample, such as the diploid genomic DNA, used for generating the target nucleic acid at that specific SNP position has at least one copy of the SNP version that is different from the version of the reference nucleic acid used. By using all possible versions at each SNP position as reference DNA—one version at a time—to compare with (forming Holliday structure/undergoing branch migration) corresponding target nucleic, followed by HJ-HJ-binder complex isolation/purification, capture of the complex by an HJ-binder receptor, and identification by hybridization using a hybridization array, one can determine the genotype of a diploid genomic DNA sample at multiple (1-millions) SNP positions simultaneously with high specificity/accuracy.

Depending on the number of SNPs to be screened, for example, oligonucleotide arrays with low, medium or high density can be employed. The density of the oligonucelotide array may be higher than 100 probes per square centimeters, optionally higher than 1000, optionally higher than 10,000, optionally higher than 1,000,000, optionally between 100-100,000,000, and optionally between 1,000,000-80,000,000 probes per square centimeters.

Preferably, high-density oligonucleotide array, chips or larger DNA probe array wafers (from which individual chips would otherwise be obtained by breaking up the wafer) are used in one embodiment of the invention. DNA probe array wafers generally comprise glass wafers on which high density arrays of DNA probes (short segments of DNA) have been formed. Each of these wafers can hold, for example, approximately 60 million or more DNA probes that are used to recognize DNA sequences. The recognition of sample DNA by the set of DNA probes on the glass wafer takes place through the mechanism of DNA hybridization. When a DNA sample hybridizes with an array of DNA probes, the sample binds to those probes that are complementary to the sample DNA sequence. By evaluating to which probes the sample DNA hybridizes more strongly, it is possible to determine whether a known sequence of DNA is present or not in the HJ-HJ-binder complex and thereby detect the presence of a SNP.

2. Preferred Embodiments of the Present Invention

In a preferred embodiment, the method is provided for detecting nucleotide sequence variation of a target nucleic acid relative to that of a target nucleic acid. The method comprises the steps of: contacting a target nucleic acid labeled with a tag with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; detecting the presence of the tag on the target nucleic acid in the Holliday junction structure in the second complex, wherein the presence of the tag on the target nucleic acid in the Holliday junction structure in the second complex is indicative of the sequence difference between the target nucleic acid and the reference nucleic acid.

In one variation of the embodiment, the receptor for the Holliday junction-binder is immobilized to a substrate such as a solid support.

According to this variation of the embodiment, the method may further comprise the step of isolating the second complex before the step of detecting the presence of the Holliday junction structure in the second complex. The step of isolating includes, but is not limited to, immunoprecipitation, gel electrophoresis, affinity chromatography, oligonucleotide array and flowing fluid sorting.

In another preferred embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; labeling one or more strand of the target or reference nucleic acid in the first complex with a tag; contacting the first complex with a receptor for the Holliday junction-binder that specifically recognizes the Holliday junction-binder; forming a second complex between the first complex and the receptor for the Holliday junction-binder; and detecting the presence of the tag on the Holliday junction structure in the second complex.

In yet another preferred embodiment, the method comprises the steps of: contacting a target nucleic acid with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a Holliday junction-binder; contacting the first complex with a receptor for the Holliday junction-binder that specifically recognizes the Holliday junction-binder; forming a second complex between the first complex and the receptor for the Holliday junction-binder; labeling one or more strand of the target or reference nucleic acid in the second complex with a tag; detecting the presence of the tag on the Holliday junction structure in the second complex.

In yet another preferred embodiment, the method comprises the steps of: contacting a target nucleic acid labeled with a tag with a reference nucleic acid, the sequence of the reference nucleic acid being the same or differing from the target nucleic acid in one or more nucleotide positions; subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions; forming a first complex between the Holliday junction structure and a protein that specifically recognizes a Holliday junction; contacting the first complex with an antibody that specifically binds to the protein that specifically recognizes a Holliday junction; forming a second complex between the first complex and the antibody; detecting the presence of the tag on the target nucleic acid in the Holliday junction structure in the second complex, wherein the presence of the tag on the target nucleic acid in the Holliday junction structure in the second complex is indicative of the sequence difference between the target nucleic acid and the reference nucleic acid.

According to the embodiment, examples of the tag include, but are not limited to, biotin, digoxigenin, fluorescent molecule (e.g., fluorescin and rhodamine), chemiluminescent moiety (e.g., luminol), coenzyme, enzyme substrate, radio isotopes, a particle such as latex or carbon particle, nucleic acid-binding protein, and polynucleotide.

According to one variation of the embodiment, the antibody that specifically binds to the protein that specifically recognizes a Holliday junction is immobilized to a substrate such as a solid support (a well of a microplate).

When the tag is biotin, the method may further comprise: contacting the second complex with an agent that comprises streptavidin conjugated to an enzyme such as alkaline phosphatase, perioxidase, or urease.

In yet another preferred embodiment, a high-throughput ELISA platform in a multi-well format is provided This platform allows users to process multiple samples simultaneously, with minimal requirements for expensive instrumentation and labor. For example, equipped with a PCR thermal cycler, a user can easily process a large number of samples for various purposes such as genotyping, SNP profiling and analysis of mutation with high accuracy and efficiency. As will be shown in the examples below, this platform enables a user to quickly and easily detect the presence of two point mutations in the YMDD motif of the Hepatitis B virus (HBV) polymerase: YVDD and YIDD.

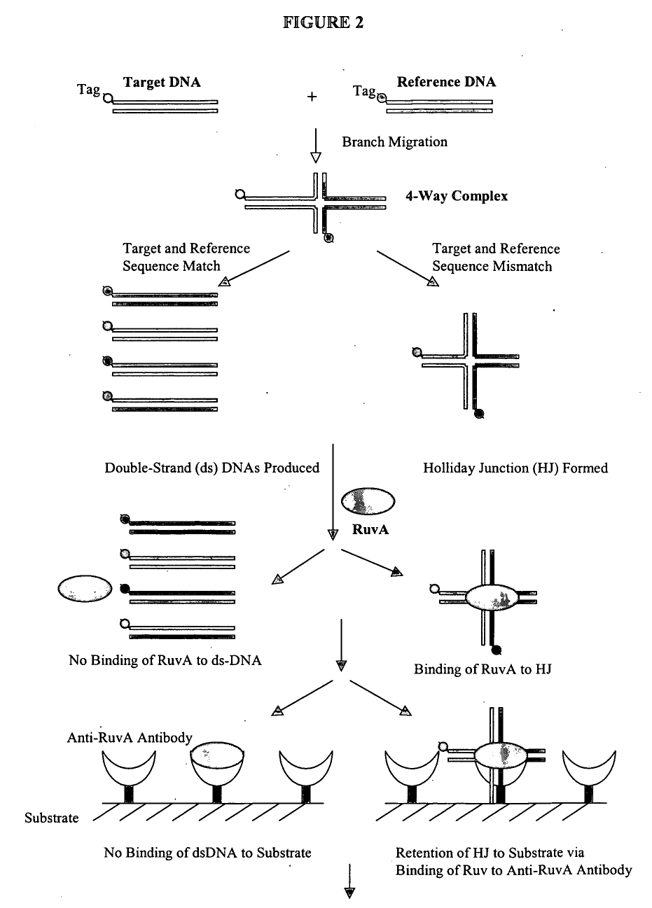

FIG. 2 is a flowchart outlining this embodiment. As illustrated in FIG. 2, a DNA sample of interest (“Target DNA”) is amplified along with a reference DNA of known genotype using specially designed primers. In this embodiment, the target DNA has a strand labeled with a tag such as biotin. As also illustrated in FIG. 2, the reference DNA may optionally be labeled with a tag the same or different from the tag on the target DNA. Alternatively, only the reference nucleic acid is labeled with a tag. The tag may be labeled before, during or after the formation of the Holliday junction.

For example, to detect two different point mutations due to SNP in the YMDD motif of the HBV polymerase: YVDD and YIDD, three different reaction tubes for each viral DNA sample may be prepared: one in which the target DNA is mixed with Reference DNA M; a second tube in which the target DNA is mixed with for Reference V; and a third tube in which the target DNA is mixed with for Reference I. All reaction tubes are preferably treated identically throughout the procedure.

Referring to FIG. 2, during branch migration step, a unique structure known as a Holliday junction forms at the SNP site. If the target and reference DNA duplexes share the same genotype, the Holliday junction resolves into double-stranded DNA; if the genotypes differ, the Holliday junction stabilizes.

Still referring to FIG. 2, the next step is detecting the stabilized Holliday junctions. An example of a Holliday junction-binder, RuvA protein, which specifically binds to Holliday junctions, is mixed with samples containing the target and reference DNA. The mixture can be incubated in a multi-well microplate (e.g., 96-well) with each well precoated with an anti-RuvA monoclonal antibody (or anti-RuvA polyclonal antibodies or anti-serum). As illustrated in FIG. 2, only the DNA strands that form a Holliday junction would be retained by the well via the binding interaction between RuvA and anti-RuvA antibody; and the double stranded DNA would not be retained after washing the wells with a buffer. Since the DNA strands on the Holliday junction have a biotin tag, upon addition of streptavidin-HRP and the substrate for HRP to the wells, the Holliday junction bound to the wells can be detected through observation or measurement of the color change as a result of enzymatic reaction of HRP. Because only the target DNA containing one or more nucleotides different from the reference DNA would form a Holliday junction under branch migration conditions, the detection of Holliday junction bound to the wells is indicative of a genotype difference.

In the application of the embodiment to detect two different point mutations due to SNP in the YMDD motif of the HBV polymerase: YVDD and YIDD, the following results would be expected as the three side-by-side reactions with References M, V and I are compared. For the wild-type genotype (YMDD): strong color reaction in V and I reference wells; for the V-for-M substitution (YVDD): strong color reaction in M and I reference wells; and for the I-for-M substitution (YIDD): strong color reaction in M and V reference wells.

As will be described in detail in the EXAMPLE section below, in the application of the embodiment to the detection of two different point mutations due to SNP in the YMDD motif of the HBV polymerase: YVDD and YIDD, a biotin-tagged forward primer, three reference DNAs, and two different reverse primers are provided to facilitate the formation and detection of the Holliday junction. The user may only need to provide his/her test DNA from which the target DNA is generated, e.g., by PCR. The ELISA results can be read using any plate reader.

Described below are preferred conditions for detecting SNPs in the YMDD motif of the HBV polymerase. The conditions may be adjusted according to the nature and purpose of the practice of the present invention.

In this embodiment, PCR amplification may be used for generating the target DNA from a sample containing HBV. HBV viral DNA may extracted from a serum sample of an infected individual using a commercial viral DNA extraction kit (e.g., Roche's High Pure Viral Nucleic Acid Kit (cat. # 1 858 874)). Control reactions may be set up to quality of the assay. For a negative control reaction, PCR-grade water may be used to replace the viral DNA. An internal positive control reaction may also be run by using specifically designed internal control primers to amplify a highly conserved region of the small HBV surface protein. A positive signal will be generated for all HBV genotypes as long as the sample contains a detectable amount of HBV DNA.

The target DNA may be PCR-amplified from HBV RNA-dependent DNA polymerase gene. For each sample, two PCR reactions may be set up: one with the target primers, and other with internal control primers. The target primers will amplify the HBV RNA-dependent DNA polymerase gene. The internal control primers will amplify a highly conserved region of the small HBV surface protein, so a positive signal for all HBV genotypes can be observed as long as the sample contains a detectable amount of HBV DNA. In this example, because the forward primer is biotinylated, the resulting DNA will also be biotinylated.

Typical PCR amplification conditions are as follows:
95° C. for 10 minutes (Activate Taq enzyme)
45 cycles of the following three steps:
−94° C. for 15 sec (denaturation)
58° C. for 23 sec (re-annealing)
72° C. for 45 sec (extension)

During the branch migration process, as illustrated in FIG. 2, a Holliday Junction structure is formed between the target DNA and reference DNA that differ from each other in one or more nucleotide positions. Specifically, aliquots of the target PCR reaction is mixed with Reference M, I, and V DNA, respectively. An aliquot of the internal control PCR reaction is mixed with Reference C DNA. Preferably, the resulting mixtures are incubated at 95° C. for 2 min and at 65° C. for 30 min. Branch migration of each mixture of target and reference amplicons will lead to Holliday Junction formation if the target amplicon has a different genotype from the reference DNA; and no Holliday junction will be formed if the target amplicon has the same genotype as the reference.

The Holliday junction formed can be detected by an ELISA assay. Specifically, RuvA protein is added to the branch migration product. Optionally, RuvA protein may be diluted with a RuvA dilution buffer. For each branch migration product, the RuvA protein may be mixed with branch migration product at a suitable ratio and incubated for 5 min at room temperature. Background correction may be performed by mixing RuvA protein with an ELISA reference. For example, a biotinylated double-stranded DNA can be used as an ELISA reference to correct background on a plate reader. If this reference gives a particular absorbance at 450 nm, the value can be substracte from the reading for each sample.

The mixture is then incubated in microplate wells precoated with anti-RuvA antibody. Preferably, the 96-well microplate precoated with anti-RuvA antibody is shipped with buffer in the wells. The buffer can be removed by pipetting or aspirating wells. The RuvA-branch migration product mixture may be mixed with a buffer (e.g., 1×TBS) in the well. As an ELISA control, the RuvA-dsDNA reference mixture is added to separate wells of the microplate. The microplate may be incubated for 1 hr at room temperature.

Following the incubation, each well may be washed with TBS buffer. Streptavidin-HRP Conjugate can then be added to each well and incubate for 1 hr at room temperature. Afterwards each well may be washed with TBS buffer before TMB Substrate Solution for HRP is added to each well. The microplate can be incubated for 20 min at room temperature. After addition of the Stop Solution for HRP reaction to each well, optical absorption at 450 nm can be measured using a plate reader.

If the target amplicon and the reference sequences differ, RuvA will bind to the resulting Holliday junction and the RuvA-HJ complex will be retained to the well of microplate precoated with anti-RuvA antibody. Since the DNA strands of the Holliday junction contain a biotin tag, the Holliday junction retained by the microplate can be labeled with streptavidin-HRP. A color change will result when substrate solution for HRP is added. If the target amplicon and the reference DNA share the same genotype, RuvA will not bind and no color change will result.

According to the present invention, a kit is also provided for the detection of SNP or mutation in target nucleic acid. Typically, the kit may contain primers and reference DNA for sequence-specific detection, convenient premixed reagents for reactions such as branch migration, and a microplate precoated with anti-RuvA antibody. Optionally, specifically designed primers for reference DNA may also be provided in lieu of ready-made reference DNA. Optionally, the kit may further contain RuvA protein, RuvA dilution buffer, streptavidin-HRP conjugate, TMB substrate solution for HRP, stop solution for HRP reaction, or concentrated TBS buffer.

Preferably, the unopened kit is stable at specified storage conditions until the expiration date printed on the label. Different storage conditions may be applied to different components in the kit. For example, it is preferred that the primers and reference DNA, the microplate precoated with anti-RuvA antibody, RuvA protein and RuvA dilution solution are stored at −20° C.; and streptavidin-HRP conjugate, TMB substrate solution for HRP, stop solution for HRP reaction, and concentrated TBS buffer are stored at 4° C.

It is also preferred not to repeatedly freeze and thaw the components. To avoid contamination, primers for generating the internal control and the target DNA, as well as PCR reaction mix, and PCR-Grade H₂O should be kept in a separate place from the other reagents.

### EXAMPLE

1. Formation and Detection of Allele-Specific Holliday Junction

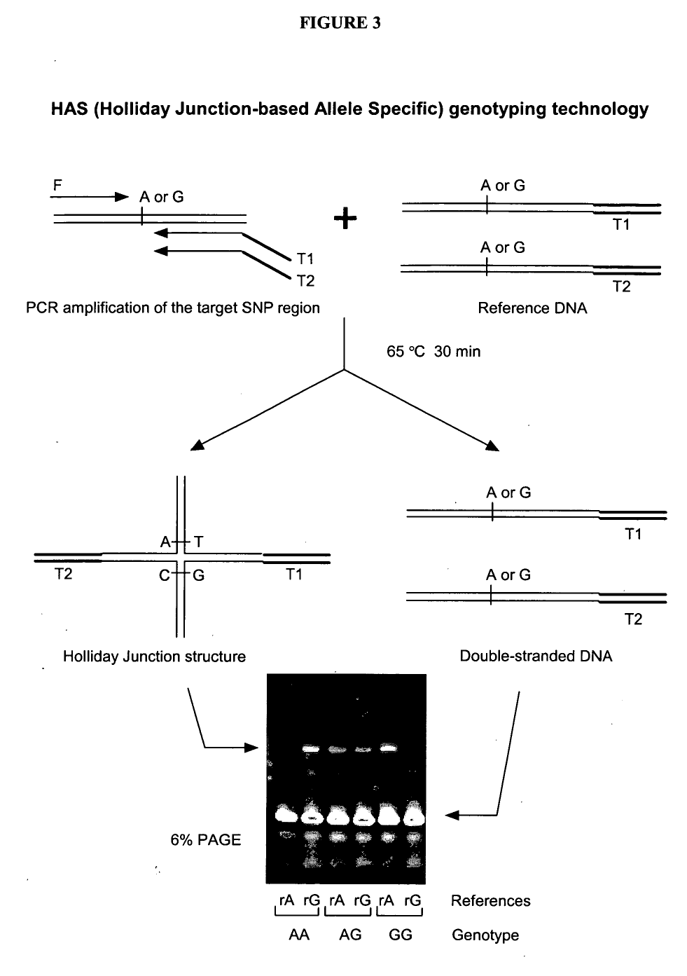

As an example, FIG. 3 shows a flow chart outlining the formation and detection of allele-specific Holliday junction by PCR amplification and branch migration inhibition. As shown in FIG. 3, the formation of Holliday junction is generally non-sequence-specific, but can occur in a temperature-dependent and allele-specific manner. For example, as shown in this figure, an allele-specific SNP of A/G variation can be detected through formation of Holliday junction by using the method of present invention. More generally, if there is mismatch at the SNP site between the target PCR amplicon and the reference DNA, a stable Holliday Junction structure is formed and the structure can be detected by using various methods, e.g., by gel electrophoresis (FIG. 3).

1.1 Primer Design

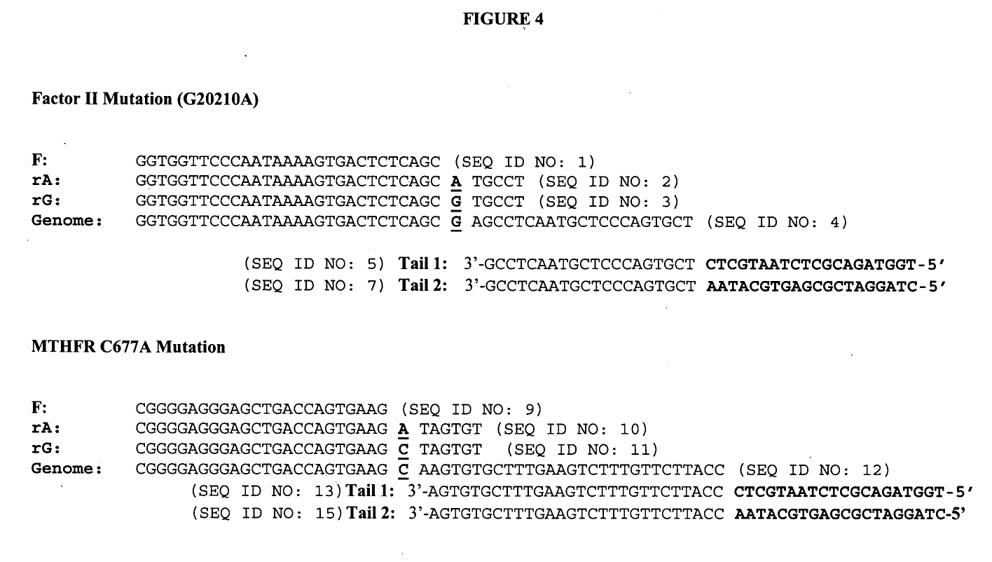

Typical primer design for amplification of the target region and reference DNA by PCR is shown in FIG. 4 (F: forwarding primer, r: reference primer, Genome: wild type human genome sequence, and Tail: tailed reverse primer (reverse complimented). As shown in FIG. 4, the reference primers have additional artificial mutation just adjacent to target SNP to stabilize the Holliday junction structure. Two tail primers have different 20-mer random sequences as “tails” to initiate the four way DNA structure formation.

1.2 Reagents, PCR Conditions, Brunch Migration and Detection on Gel Electrophresis

A improved method for Holliday junction based allele-specific genotyping is described as follows. While a basic two-step protocol requires the step to mix the reference DNAs with the target amplicon, a convenient single step protocol has also been developed. In the single step protocol, amplification of both the target and the reference DNA is performed in a single tube so that PCR and Holliday junction formation can be performed in continuous thermal cycling steps without changing tubes.

A. Two-Step Protocol (A Two-Tube System):

The following lists the components in a PCR reaction mixture to amplify the target SNP region from human genomic DNA (the PCR product (i.e., amplicon) size is typically 70-80 bp)
10 mM Tris-HCl, pH 8.3
2 mM MgCl2
50 mM KCl
200 ng/μl BSA
200 nM each dNTP
1 μM forward primer (salt free)
0.25 μM reverse tailed primer T1 (PAGE purified)
0.25 μM reverse tailed primer T2 (PAGE purified)
0.1 ng/μl genomic DNA
0.2 0.025 U/μl Hot-Start Taq DNA polymerase (Taq-Gold)
Total: 15 μl/SNP detection

Thermal-cycle condition for the above reaction mixture is as follows.
95° C. 10 min (Activate the Taq-Gold)
94° C. 15 sec
45 cycles
58° C. 23 sec
72° C. 45 sec

Condition for branch migration is described as follows:
Mix 5 μl of the target PCR reaction product with 5 μl of Ref1
Mix 5 μl of the target PCR reaction product with 5 μl of Ref2
(the two reference DNA, Ref1 and Ref2, are provided in the kit)

Incubate the two mixtures as follows:
95° C. 2 min
65° C. 30 min

Holliday Junction formed after branch migration can be detected by gel electrophoresis (e.g., polyacrylamide gel electrophoresis (PAGE)) by following this protocol:

For 6% PAGE,
Pipet 5 μl of Branch Migration product and mix it with 1.25 μl 5×Loading Buffer (Invitrogen Inc. San Diego, Calif.)
Load the mixture in a well of a 6% pre-cast polyacrylamide gel (Pre-cast TBE gel: Invitrogen Inc. San Diego, Calif.)
Run the gel at ˜200 Volts for 20 min
Dilute SYBR Gold (Molecular Probes, Eugene, Oreg.) 10000-fold in TBE buffer to make a 1× staining solution
Stain the gel for 10 min and photograph with Polaroid 667 films

For 3% agarose gel electrophoresis, a standard agarose gel protocol can be used.

B. Single Step Protocol (One Tube System):

The following lists the components in a PCR reaction mixture for amplifing the target SNP region and a reference DNA fragment from human genomic DNA (PCR product size is typically 70-80 bp):
10 mM Tris-HCl, pH 8.3
2 mM MgCl2
50 mM KCl
200 ng/μl BSA
200 nM each dNTP (use dUTP instead of dTTP)
0.8 μM forward primer (salt free)
0.2 μM reference primer, ref1 or ref2 (salt free)
0.25 μM reverse tailed primer T1 (gel purified)
0.25 μM reverse tailed primer T2 (gel purified)
0.1 ng/μl genomic DNA
0.025 U/μl Hot-Start Taq DNA polymerase (Taq-Gold)

Two reaction mixtures (Ref1 and Ref2: total 10 □l each) were prepared for one SNP detection.

Thermal-cycling condition for both PCR and Branch Migration is as follows:
95° C. 10 min (Activate the Taq-Gold)
94° C. 15 sec
45 cycles
58° C. 23 sec (PCR)
72° C. 45 sec
95° C. 2 min+65° C. 30 min (Branch Migration)

Holliday Junction formed after branch migration can be detected by gel electrophoresis (e.g., polyacrylamide gel electrophoresis (PAGE)) by following this protocol:

For 6% PAGE,
Pipet 5 μl of Branch Migration product and mix it with 1.25 μl 5× Loading Buffer (Invitrogen Inc. San Diego, Calif.)
Load the mixture in a well of a 6% pre-cast polyacrylamide gel (Pre-cast TBE gel: Invitrogen Inc. San Diego, Calif.)
Run the gel at ˜200 Volts for 20 min
Dilute SYBR Gold (Molecular Probes, Eugene, Oreg.) 10000-fold in TBE buffer to make a 1× staining solution
Stain the gel for 10 min and photograph with Polaroid 667 films

For 3% agarose gel electrophoresis, a standard agarose gel protocol can be used.

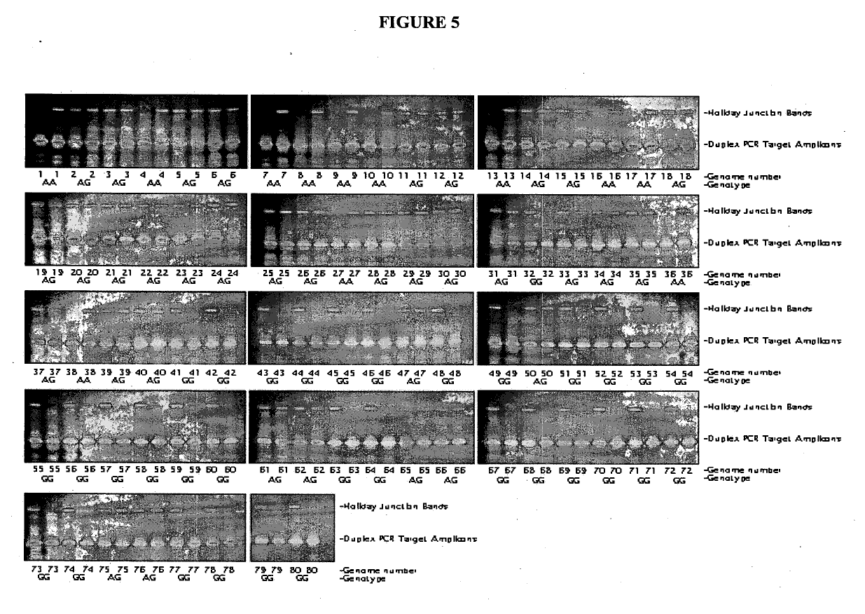

FIG. 5 shows that a PAGE-based Holliday junction allele-specific genotyping method was used to genotype the HFE C282Y mutation on 80 genomic DNA samples supplied by UC Davis Medical Center. Genotype for each genomic DNA sample is judged by presence or absence of the Holliday Junction band on refA lane (left) and refG lane (right).

2. Detection of Mutation in Human Factor V by Holliday Junction-Based ELISA

2.1 RuvA-Based Detection of Holliday Junction

In prokaryotes, RuvA processes a Holliday junction which is the universal DNA intermediate of homologous recombination. RuvA protein specifically and tightly binds to a Holliday junction. The binding is not sequence-specific, but is highly specific for the four-way structure of DNA. It is believed that Holliday junction-based genotyping with RuvA can be applied to detection of virtually all SNPs because of the sequence non-specific binding property of RuvA.

2.2 Gel Shift Binding Assay of RuvA-Bound Holliday Junction

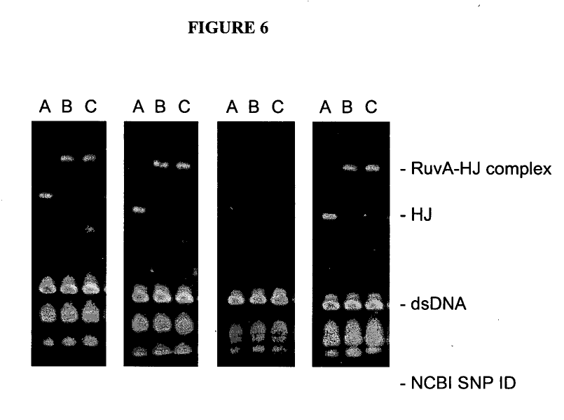

Binding profile of RuvA to PCR-based synthetic Holliday junction was assayed by gel shift on 6% PAGE. Primers for randomly picked up four SNPs were designed and the Holliday junction formation was performed using human genomic DNA as templates. Resulted Holliday junction structures were mixed with RuvA protein and analyzed on PAGE (FIG. 6). Briefly, 5 ul of Holliday junction product corresponding to the SNP ID indicated under the gel picture was mixed with 1 ul of 10 uM RuvA protein at room temperature and analyzed on 6% PAGE (lane A: no RuvA control, lane B: incubated with RuvA for 5 min, and lane C: incubated with RuvA over night). As shown in FIG. 6, the clear gel shift bands of Holliday junction indicate that RuvA binds specifically to synthetic Holliday junction and the binding is not sequence-specific. No shift of dsDNA bands was observed, indicating that the binding was highly specific for Holliday junction.

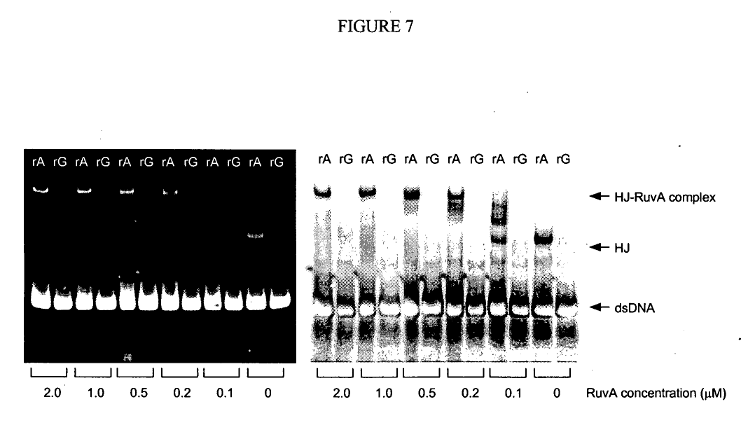

Effects of RuvA concentration on the HJ binding were also tested by the gel shift assay. Briefly, biotinylated PCR primer was used to amplify Human Factor V mutation (A1691G) target region, and the resulted Holliday junction structure was incubated with RuvA at various concentrations for 5 min at room temperature and the results were shown in FIG. 7 (left panel: gel shift observed on 6% PAGE stained with SYBR Gold; right panel: the gel transferred to membrane, and detected with chemiluminescence). As shown in FIG. 7, 0.2 uM RuvA is a sufficient concentration for binding to all Holliday junction structure in the genotyping assay. The Holliday junction formation and specific RuvA binding were not inhibited by biotinylation of PCR product, indicating that biotin labeled Holliday junction DNA can be detected with high-sensitivity.

2.3 ELISA Detection of SNP in Human Factor V Gene Based on Formation of Holliday Junction

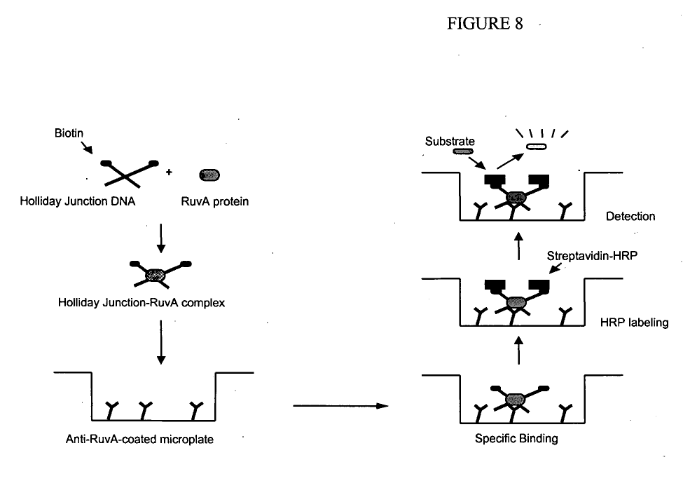

To detect stabilized Holliday junctions, the RuvA protein was added to the branch migration products generated by targeting the A/G mutation of human Factor V gene using specifically designed primers shown below. The mixture was then incubated in a 96-well microplate with each well precoated with anti-RuvA antibody. The presence of Holliday junctions, which is indicative of a genotype difference, can then be visualized by a color reaction. For each sample, the two side-by-side reactions with References A and G are compared. FIG. 8 is a schematic illustration of the process described above. As illustrated in FIG. 8, RuvA protein is added to the branch migration product. The mixture is then incubated in microplate wells precoated with anti-RuvA antibody, and biotin-labeled branch migration productions are also labeled with streptavidin-HRP. If the target amplicon and reference sequences differ, RuvA will bind to the resulting Holliday junction. For the biotin-labeled Holliday junction structure bound to the anti-RuvA antibody via RuvA, detection of the biotin on the structure (and thus the formation of the Holliday junction) can be achieved by measuring a color change upon addition of a substrate solution such as streptavidin-HRP. If the target amplicon and reference share the same genotype, RuvA will not bind and no color change will result.

The following is a list of the primers designed for genotyping A/G mutation of human Factor V gene.

(SEQ ID NO:17)

F5-F (Forward)
5′Biotin-GAGCAGATCCCTGGACAGGC

(SEQ ID NO:18)

F5-rA (Reference A)
GAGCAGATCCCTGGACAGGCATGGAA

(SEQ ID NO:19)

F5-rG (Reference G)
GAGCAGATCCCTGGACAGGCGTGGAA

(SEQ ID NO:20)

F5R3T1 (Reverse 1)
ACCATCGTCGAGATTACGTCTTCAAGGACAAAATACCTGTATTCC

(SEQ ID NO:21)

F5R3T2 (Reverse 2)
GATCCTAGGCCTCACGTATTTTCAAGGACAAAATACCTGTATTCC

The single step PCR/branch migration protocol as described in section 1.2 above was performed for the target region of human Factor V gene.

An ELISA plate was prepared by using the following protocol:
Add 100 ul of 1 ug/ml (or various concentration) Anti-RuvA monoclonal antibody (in PBS buffer) to each well of 96-well ELISA plate;
Incubate the plate at 4° C. overnight;
Remove the antibody solution completely;
Wash wells with 400 ul TBS;
Add 200 ul BSA-TBS blocking buffer;
Incubate at room temperature for 2 hours;
Remove the blocking buffer completely; and
Add 200 ul TBS and store at 4° C.

Detection of Hollidayjunction formation by ELISA was performed by using the following protocol:
For each branch migration product, mix 2 μl (or various amount) of 2.6 μM RuvA with 10 μl of branch migration product (step 1);
Incubate for 5 min at room temperature;
Add 80 μl of PCR buffer or TBS to each well of the 96-well microplate;
Add 1-5 μl of each RuvA-Branch Migration mixture (from step 1) to separate wells of the microplate, mix by pipetting, and incubate for 1 hr at room temperature;
Wash each well 5 times with 200 μl of diluted TBS buffer;
Dilute 1000× Streptavidin-HRP Conjugate 1:1000 with 1×TBS;
Add 100 μl of 1× Streptavidin-HRP Conjugate to each well, and incubate for 1 hr at room temperature;
Wash each well 5 times with 200 μl of 1×TBS buffer;
Add 100 μl of TMB Substrate Solution to each well, and incubate for 20 min at room temperature;
Add 100 μl of Stop Solution to each well; and
Detect A450 using a plate reader, or judge color reaction by eyes.

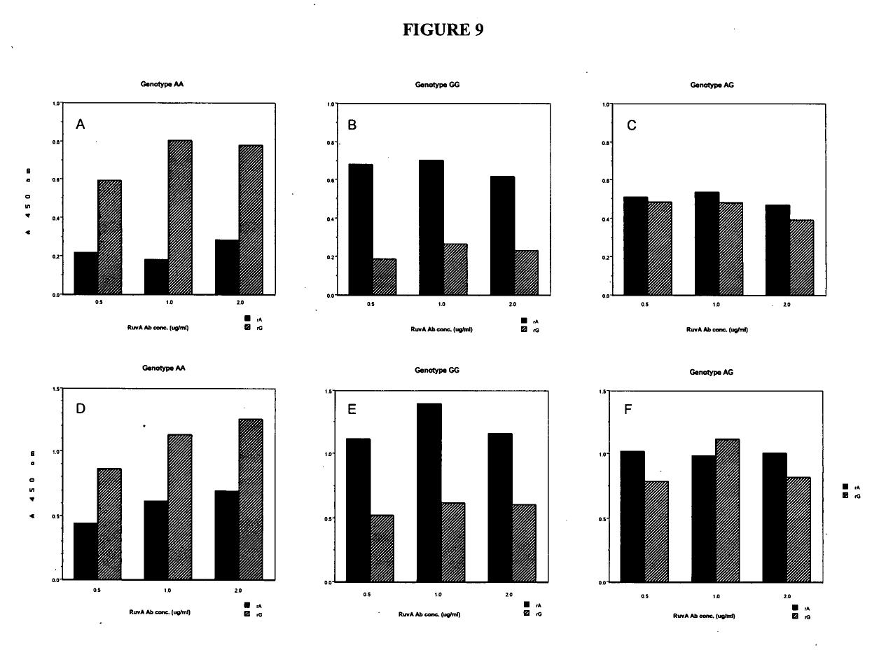

The above ELISA was further modified and optimized based on experiments on Anti-RuvA antibody concentration, RuvA concentration and sample amount on ELISA format and the results are shown in FIGS. 9-11. The optimization of anti-RuvA antibody concentration and sample amounts was based on human Factor V genotyping system described above. All three genotypes (AA, AG and GG) were amplified from Human genomic DNA using specific primers (see above) followed by branch migration, and the samples were incubated with 0.5 μM RuvA. This concentration of RuvA is sufficient to bind all Holliday junction structure in branch migration sample (see FIG. 7). One μl (A, B and C) or 5 μM (D, E and F) of the sample mixtures were then added to 80 μl PCR buffer in each wells of ELISA plate.

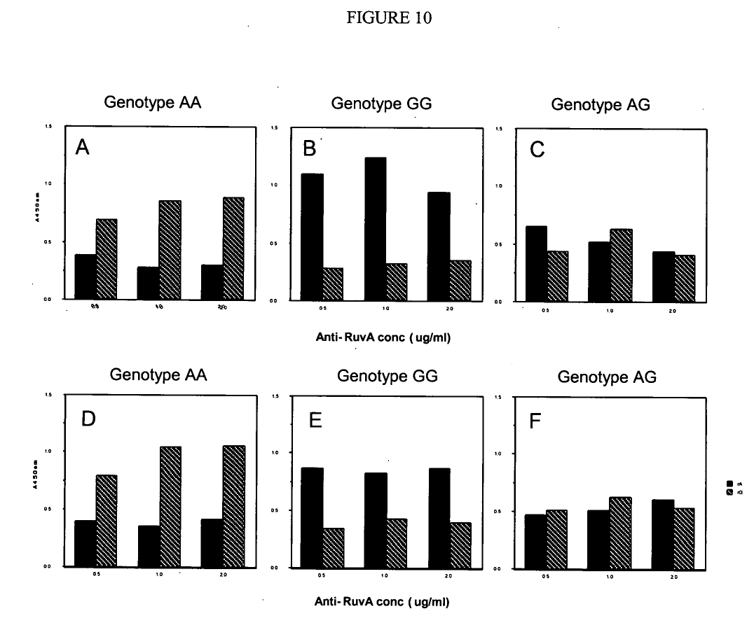

FIG. 7 shows the results of Holliday junction formation due to the A/G mutation in human Factor V that was detected by ELISA of the present invention. FIG. 10 shows the effect of different branch migration temperatures on the ELISA sensitivity at lower RuvA concentrations. Briefly, the Holliday junction products for all three genotypes (AA, AG and GG) were prepared as in FIG. 9, and the samples were incubated with 0.2 μM RuvA. Branch migration temperature was 62° C. (A, B and C) or 64° C. (D, E and F). One μl of the sample mixture was added to 80 μl PCR buffer in each wells of ELISA plate. Based on these results, the following condition was selected for further optimization of the ELISA:
Branch migration temperature: 62° C.
RuvA concentration: 0.5 μM
Anti-RuvA concentration for plate preparation: 1.0 μg/ml; and
Branch migration sample amount: 1 μl

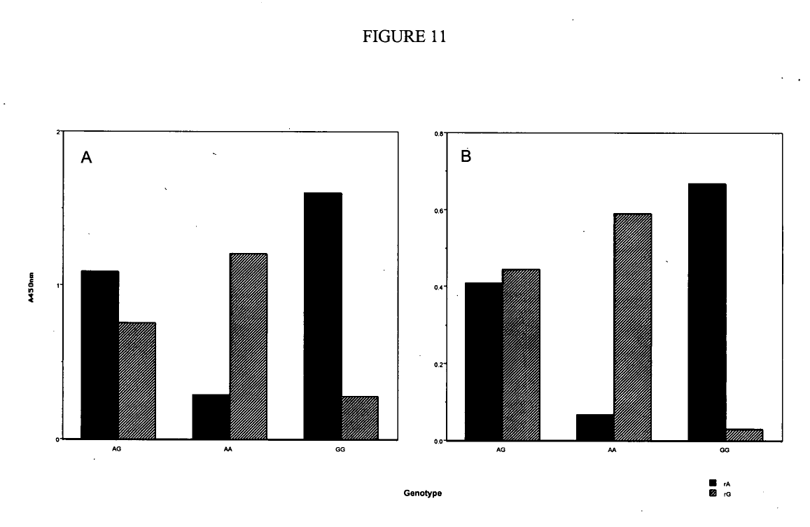

FIG. 11 shows the effect of binding buffer on background level in the ELISA. Briefly, TBS and PCR buffer (see section 1.2) were used for RuvA-anti RuvA binding buffer, and the background level was compared. Holliday junction samples for all three genotypes of Factor V were prepared as in FIG. 9. After branch migration, 2 μl of 2.6 μM RuvA was mixed with the branch migration sample, and 1 μl of the mixture was added to 80 μl PCR buffer (A) or TBS (B) in each well of anti-RuvA antibody-coated ELISA plate. Detection was performed as described in section 2.3 above. FIG. 11 clearly shows that TBS buffer gives lower background and thus TBS was selected for further development.

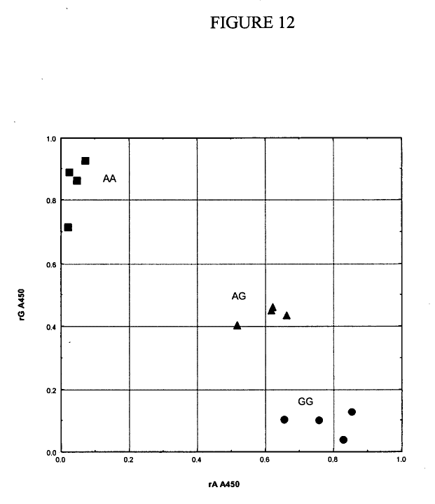

The three genotypes in each sample were analyzed using the methods described above and the results are shown in FIG. 12. As shown in FIG. 12, the three genotypes are clearly discriminated.

3. Detection of Mutation in Human Factor II and MTHFR by Holliday Junction-Based ELISA

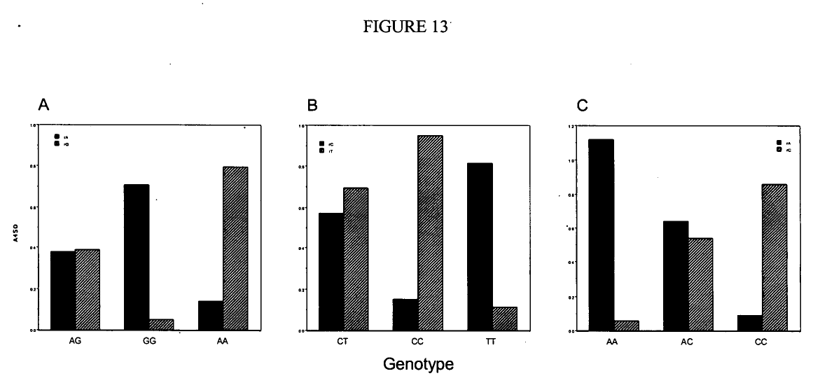

With slight modifications, the Holliday junction-based ELISA developed above was successfully applied to detection of mutation in human Factor II and MTHFR mutation and the results are shown in FIG. 13. The single step PCR/branch migration protocol (see section 1.2 above) was performed for human Factor II G20210A mutation (A), MTHFR C677T (B) and A1298C(C) mutations using specific primers. Ten μl of the branch migration products of each sample were mixed with 2 μl of 2.6 μM RuvA, and 3 μl (A), 1.5 μl (B) or 1.0 μl (C) of the mixtures were analyzed using ELISA as described in panel B of FIG. 11.

4. Single-Wash ELISA

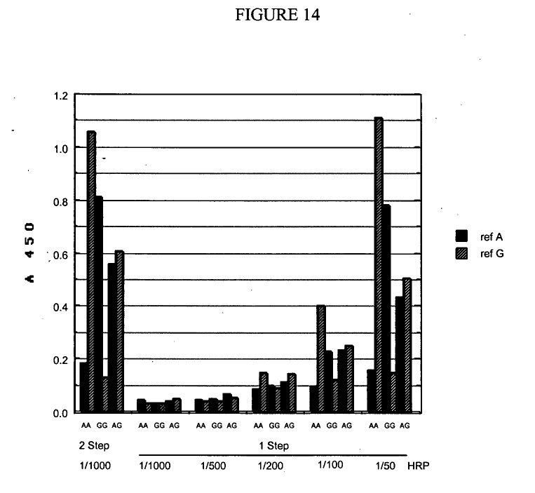

To shorten the detection time for large-scale sample handling, the antibody binding step and HRP labeling step was performed concurrently, resulting in a single-wash ELISA. FIG. 14 shows the effect of HRP concentration on efficiency of detection in such a single-wash ELISA as compared to that in a standard protocol involving 2 separate steps of antibody binding and HRP labeling (see section 3 above). Holliday junction formation was performed under the same condition as in panel B of FIG. 11. Background level was not corrected in FIG. 14. As shown in FIG. 14, when the HRP concentration of single step protocol was 20 times higher (1/50 HRP) than that (1/1000 HRP) of the standard protocol, the modified protocol was almost equally efficient compared to the standard protocol. The details of the modified protocol is as follows:
Add 2 μl of 2.6 μM RuvA protein to 10 μl of Brunch Migration product of Factor V, incubate 5 min at room temp;
Add 80 μl HRP solution (diluted 1:000-1:50 in TBS) to each well of anti-RuvA pre-coated 96-well microplate;
Mix 1 μl of RuvA-Brunch Migration mixture to each well of the microplate. Incubate 1 h at room temp;
Wash 5 times with 200 μl TBS buffer;
Add 100 μl of substrate solution to each well;
Incubate 20 min at room temp;
Add 100 μl of stop solution to each well; and
Detect A450 with plate reader or judge by eyes.
   5. Genotyping by ELISA Using Recombinant E. coli RuvA

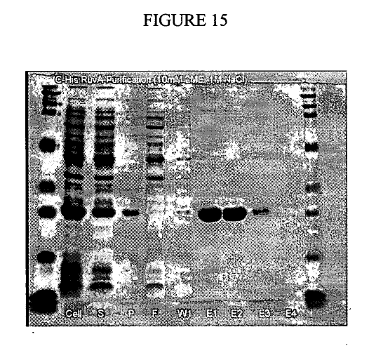

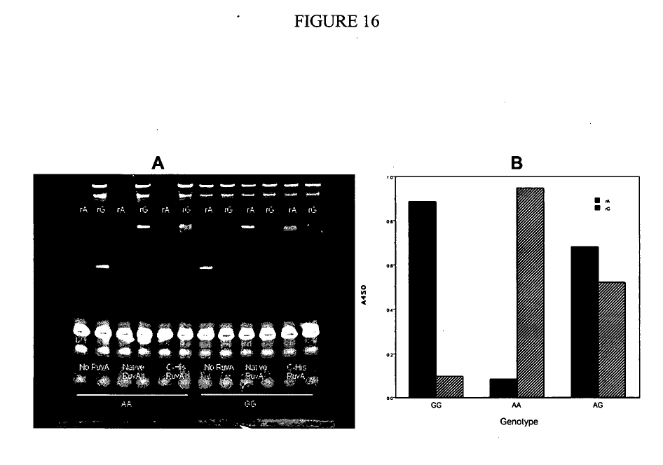

Reported 3-dimensional structure of a complex formed between E. coli RuvA and a Holliday junction indicates that the two domains at the N-terminus of RuvA play key roles in binding to the Holliday junction and the C-terminal domain has no direct interaction with the Holliday junction. To test the effect of a tagged C-terminus on the binding affinity of RuvA and to provide a more economic resource for large production of RuvA, a recombinant RuvA tagged with 6×His at the C-terminus was constructed and purified from E. coli culture. The His-tagged RuvA was expressed and purified with a very high yield (˜40 mg purified protein from 1 liter of host E. coli culture). FIG. 15 shows a purification profile of C-terminal 6×His-tagged RuvA protein using a Ni-NTA column. Second elution fraction (E2) contains 4.62 mg/ml protein (5 ml), and was demonstrated to be fully functional under the condition of Holliday-junction based ELISA for genotyping (FIG. 16). As shown in FIG. 16, the function of His-tagged RuvA was compared with that of no His-tagged recombinant RuvA using a gel shift assay (panel A). The assay condition was the same as in FIG. 6. The His-tagged protein was also tested in the Holliday junction-based ELISA for genotyping (panel B). All conditions for the ELISA was the same as that in panel B of FIG. 11.

6. Holliday Junction-Based ELISA for Detection of Viral Mutation

The Holliday junction-based ELISA developed above was successfully used to detect viral mutation in HBV.

6.1 Primer Design and Reaction Condition

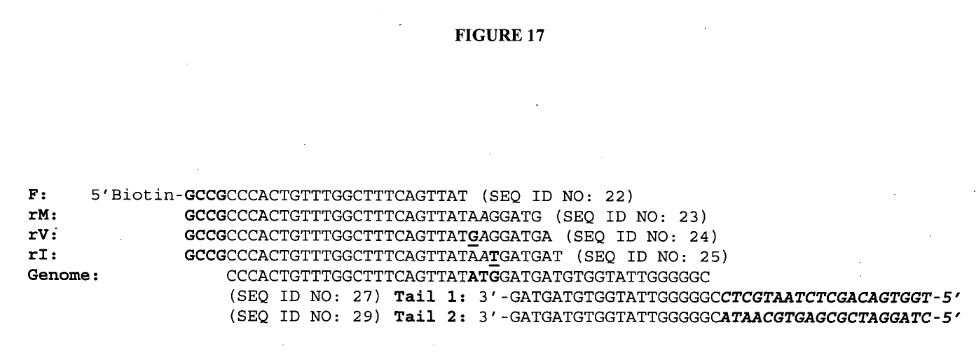

In this example, the ELISA of the present invention was applied to detect point mutation in the YMDD motif of HBV. It is known that prolonged lamivudine treatment is often associated with the emergence of drug-resistant HBV species. Lamivudine-resistant species result from specific amino-acid substitutions in the HBV-encoded polymerase. The most common mutations occur in the YMDD motif, where either valine (codon GTG) or isoleucine (codon ATT) is substituted for methionine (codon ATG). Specific primers for this mutation region were designed and shown in FIG. 17 (F: forwarding primer, r: reference primer, Genome: HBV genome sequence, and Tail: tailed reverse primer (reverse complimented)).

In this example, the two step PCR/branch migration protocol described in section 1.2 was used to obtain Holliday junction structures.

6.2 Detection of Mutation-Specific Holliday Junction Formation on Page

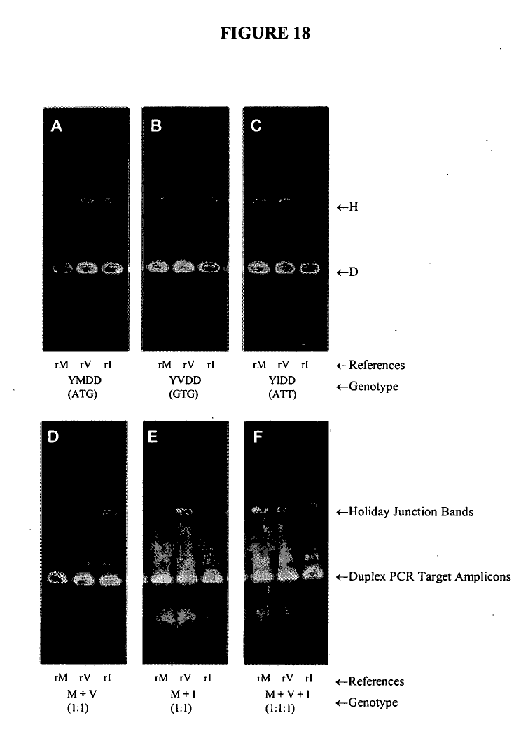

Because two different point mutations occur at same codon in the HBV polymerase gene, three references were used to discriminate all types of mutation. Typical results obtained by running PAGE are shown in FIG. 18 (panel A: Wild type YMDD, strong HJ bands on Ref. V and Ref. I lanes; panel B: YVDD mutant, strong HJ bands on Ref. M and Ref. I lanes; panel C: YIDD mutant, strong HJ bands on Ref. M and Ref. V lanes; panel D: Mixture of YMDD and YVDD, weak HJ bands on Ref. M and Ref. V lanes, strong HJ band on Ref. I lane; panel E: Mixture of YMDD and YIDD, weak HJ bands on Ref. M and Ref. I lanes, strong HJ band on Ref. V lane; and panel F: Mixture of YMDD, YVDD and YIDD, HJ bands on all three reference lanes).

6.3. Detection of HBV YMDD Mutation by Holliday Junction-Based ELISA

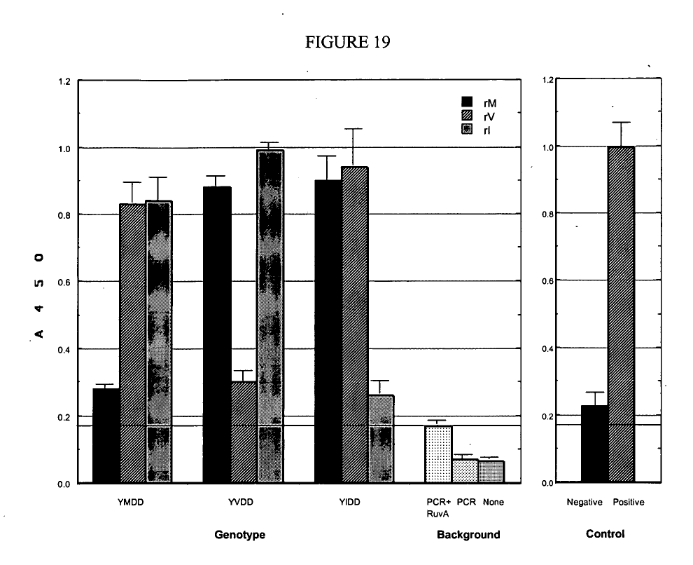

To detect mutation in HBV YMDD motif, a total of 15 DNA samples (five samples for each genotype) were processed according to the two-step PCR/branch migration (section 1.2) and two-wash ELISA protocol (section 2.3). FIG. 19 shows the averaged results. The internal control bar represents the average of all 15 internal control reactions. The background level was obtained by running a background correction of biotinylated dsDNA. An amount of 1.5 μl Holliday junction-RuvA complex was added to each well of the ELISA plate. The results indicate the Holliday junction-based ELISA assay of the present invention can sensitively detect and discriminate point mutation or SNP in virus.

1. A method for detecting nucleotide sequence variation of a target nucleic acid relative to that of a reference nucleic acid, comprising the steps of:
providing a Holliday junction structure formed between a target nucleic acid and a reference nucleic acid, the reference nucleic acid differing in sequence from the target nucleic acid in one or more nucleotide positions;
forming a first complex between the Holliday junction structure and a Holliday junction-binder;
contacting the first complex with a receptor for the Holliday junction-binder that specifically recognizes the Holliday junction-binder;
forming a second complex between the first complex and the receptor for the Holliday junction-binder; and
detecting the presence of the Holliday junction structure in the second complex, wherein the presence of the Holliday junction structure in the second complex is indicative of the sequence difference between the target nucleic acid and the reference nucleic acid.

2. The method of claim 1, wherein the target nucleic acid is derived from a targeted region of a test nucleic acid contained in a sample.

3. The method of claim 2, wherein the target nucleic acid is PCR-amplified from a region of the test nucleic acid containing a SNP.

4. The method of claim 2, wherein the test nucleic acid is selected from the group consisting of double-stranded test DNA, single-stranded test DNA, test RNA, test DNA-RNA hybrid of a target gene, chromosome, plasmid, or genome of a biological material.

5. The method of claim 4, wherein the biological material is selected from the group consisting of bacteria, yeasts, viruses, viroids, molds, fungi, plants, animals, and humans.

6. The method of claim 2, wherein the sequence of the reference nucleic acid differs from the sequence of the target nucleic acid in a single nucleotide position.

7. The method of claim 1, wherein the target nucleic acid is double-stranded.

8. The method of claim 2, wherein the target nucleic acid comprises a combination of Target-Tail-1 polynucleotide and Target-Tail-2 polynucleotide, wherein the Target-Tail-1 polynucleotide comprises a first region that is substantially homologous to the target region of the test nucleic acid and a second region designated as Tail-1; the Target-Tail-2 polynucleotide comprises a first region that is substantially homologous to the target region of the test nucleic acid and a second region designated as Tail-2; and the sequence of Tail-1 and that of Tail-2 differ in one or more nucleotide positions.

9. The method of claim 8, wherein the reference nucleic acid comprises a combination of Reference-Tail-1 polynucleotide and Reference-Tail-2 polynucleotide, wherein the Reference-Tail-1 polynucleotide comprises a first region that is substantially homologous to the target region of the test nucleic acid and a second region designated as Tail-1; the Reference-Tail-2 polynucleotide comprises a first region that is substantially homologous to the target region of the test nucleic acid and a second region designated as Tail-2; and the sequence of Tail-1 and that of Tail-2 differ in one or more nucleotide positions.

10. The method of claim 9, wherein the Target-Tail-1 polynucleotide or the Target-Tail-2 polynucleotide is double-stranded.

11. The method of claim 10, wherein the Reference-Tail-1 polynucleotide or the Reference-Tail-2 polynucleotide is double-stranded.

12. A method for detecting nucleotide sequence variation of a target nucleic acid relative to that of a reference nucleic acid, comprising the steps of:
contacting a target nucleic acid with a reference nucleic acid, the sequence of the reference nucleic acid being the same or differing from the target nucleic acid in one or more nucleotide positions;
subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions;
forming a first complex between the Holliday junction structure and a Holliday junction-binder;
contacting the first complex with a receptor for the Holliday junction-binder that specifically recognizes the Holliday junction-binder;
forming a second complex between the first complex and the receptor for the Holliday junction-binder; and
detecting the presence of the Holliday junction structure in the second complex, wherein the presence of the Holliday junction structure in the second complex is indicative of the sequence difference between the target nucleic acid and the reference nucleic acid.

13. The method of claim 12, wherein the target nucleic acid is derived from a targeted region of a test nucleic acid contained in a sample.

14. The method of claim 13, wherein the target nucleic acid is PCR-amplified from a region of the test nucleic acid containing a SNP.

15. The method of claim 13, wherein the test nucleic acid is selected from the group consisting of double-stranded test DNA, single-stranded test DNA, test RNA, test DNA-RNA hybrid of a target gene, chromosome, plasmid, or genome of a biological material.

16. The method of claim 15, wherein the biological material is selected from the group consisting of bacteria, yeasts, viruses, viroids, molds, fungi, plants, animals, and humans.

17. The method of claim 13, wherein the sequence of the reference nucleic acid differs from the sequence of the target nucleic acid in a single nucleotide position.

18. The method of claim 12, wherein the target nucleic acid is double-stranded.

19. The method of claim 13, wherein the target nucleic acid comprises a combination of Target-Tail-1 polynucleotide and Target-Tail-2 polynucleotide, wherein the Target-Tail-1 polynucleotide comprises a first region that is substantially homologous to the target region of the test nucleic acid and a second region designated as Tail-1; the Target-Tail-2 polynucleotide comprises a first region that is substantially homologous to the target region of the test nucleic acid and a second region designated as Tail-2; and the sequence of Tail-1 and that of Tail-2 differ in one or more nucleotide positions.

20. The method of claim 19, wherein the reference nucleic acid comprises a combination of Reference-Tail-1 polynucleotide and Reference-Tail-2 polynucleotide, wherein the Reference-Tail-1 polynucleotide comprises a first region that is substantially homologous to the target region of the test nucleic acid and a second region designated as Tail-1; the Reference-Tail-2 polynucleotide comprises a first region that is substantially homologous to the target region of the test nucleic acid and a second region designated as Tail-2; and the sequence of Tail-1 and that of Tail-2 differ in one or more nucleotide positions.

21. The method of claim 20, wherein the Target-Tail-1 polynucleotide or the Target-Tail-2 polynucleotide is double-stranded.

22. The method of claim 21, wherein the Reference-Tail-1 polynucleotide or the Reference-Tail-2 polynucleotide is double-stranded.

23. The method of claim 21, further comprising:
PCR-amplifying the targeted region of the test nucleic acid using
a forward primer for the targeted region of the test nucleic acid,
a first reverse primer for the targeted region of the test nucleic acid further comprising the sequence of Tail-1, and
a second reverse primer for the targeted region of the test nucleic acid further comprising the sequence of Tail-2.

24. The method of claim 12, wherein the reference nucleic acid or the target nucleic acid has a length of 20-500 nucleotides.

25. The method of claim 12, wherein the reference nucleic acid or the target nucleic acid has a length of 30-100 nucleotides.

26. The method of claim 12, wherein the reference nucleic acid or the target nucleic acid has a length of 50-80 nucleotides.

27. The method of claim 12, wherein the step of detecting the presence of the Holliday junction structure in a second complex includes detecting the presence of one or more strands of the Holliday junction by a method selected from the group consisting of colorimetric detection, fluorescence detection, chemiluminescent detection, enzymatic reaction, gel electrophoresis, mass spectroscopy, and oligonucleotide array.

28. A method for detecting nucleotide sequence variation of a target nucleic acid relative to that of a reference nucleic acid, comprising the steps of:
contacting a target nucleic acid with a reference nucleic acid, the sequence of the reference nucleic acid being the same or differing from the target nucleic acid in one or more nucleotide positions;
subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions;
forming a first complex between the Holliday junction structure and a Holliday junction-binder;
contacting the first complex with a receptor for the Holliday junction-binder that specifically recognizes the Holliday junction-binder, the receptor being immobilized to a substrate;
forming a second complex between the first complex and the receptor for the Holliday junction-binder; and
detecting the presence of the Holliday junction structure in the second complex, wherein the presence of the Holliday junction structure in the second complex is indicative of the sequence difference between the target nucleic acid and the reference nucleic acid.

29. The method of claim 28, wherein the substrate to which the receptor for Holliday junction-binder is immobilized is a solid support.

30. The method of claim 29, wherein the solid support is selected from the group consisting of a microsphere bead, a magnetic bead, a well of a culture plate, glass, membrane and fabric.

31. The method of claim 28, further comprising the step of:
isolating the second complex before the step of detecting the presence of the Holliday junction structure in the second complex.

32. The method of claim 31, wherein the step of isolating includes a method selected from the group consisting of immunoprecipitation, gel electrophoresis, affinity chromatography, oligonucleotide array and flowing fluid sorting.

33. The method of claim 31, wherein the step of detecting the presence of the Holliday junction structure in the isolated second complex includes detecting the presence of one or more strands of the Holliday junction by a method selected from the group consisting of colorimetric detection, fluorescence detection, chemiluminescent detection, enzymatic reaction, gel electrophoresis, mass spectroscopy, and oligonucleotide array.

34. A method for detecting nucleotide sequence variation of a target nucleic acid relative to that of a reference nucleic acid, comprising the steps of:
contacting a target nucleic acid labeled with a tag with a reference nucleic acid, the sequence of the reference nucleic acid being the same or differing from the target nucleic acid in one or more nucleotide positions;
subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions;
forming a first complex between the Holliday junction structure and a Holliday junction-binder;
contacting the first complex with a receptor for the Holliday junction-binder that specifically recognizes the Holliday junction-binder;
forming a second complex between the first complex and the receptor for the Holliday junction-binder; and
detecting the presence of the tag on the target nucleic acid in the Holliday junction structure in the second complex, wherein the presence of the tag on the target nucleic acid in the Holliday junction structure in the second complex is indicative of the sequence difference between the target nucleic acid and the reference nucleic acid.

35. The method of claim 34, wherein the tag is selected from the group consisting of biotin, digoxigenin, fluorescent molecule, chemiluminescent moiety, coenzyme, enzyme substrate, radio isotopes, a particle, nucleic acid-binding protein, and polynucleotide.

36. The method of claim 34, wherein the receptor for the Holliday junction-binder is immobilized to a solid support.

37. The method of claim 36, wherein the solid support is a well of a culture plate.

38. The method of claim 34, further comprising step of:
isolating the second complex before the step of detecting the presence of the Holliday junction structure in the second complex.

39. The method of claim 38, wherein the step of isolating is performed by using a method selected from the group consisting of immunoprecipitation, gel electrophoresis, affinity chromatography, oligonucleotide array and flowing fluid sorting.

40. A method for detecting nucleotide sequence variation of a target nucleic acid relative to that of a reference nucleic acid, comprising the steps of:
contacting a target nucleic acid with a reference nucleic acid, the sequence of the reference nucleic acid being the same or differing from the target nucleic acid in one or more nucleotide positions;
subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions;
forming a first complex between the Holliday junction structure and a Holliday junction-binder;
labeling one or more strand of the target or reference nucleic acid in the first complex with a tag;
forming a second complex between the first complex and the receptor for the Holliday junction-binder; and
detecting the presence of the tag on the Holliday junction structure in the second complex.

41. The method of claim 40, wherein the tag is selected from the group consisting of biotin, digoxigenin, fluorescent molecule, chemiluminescent moiety, coenzyme, enzyme substrate, radio isotopes, a particle, nucleic acid-binding protein, and polynucleotide.

42. The method of claim 40, wherein the receptor for the Holliday junction-binder is immobilized to a solid support.

43. The method of claim 42, wherein the solid support is a well of a culture plate.

44. The method of claim 40, further comprising step of:
isolating the second complex before the step of detecting the presence of the Holliday junction structure in the second complex.

45. The method of claim 44, wherein the step of isolating is performed by using a method selected from the group consisting of immunoprecipitation, gel electrophoresis, affinity chromatography, oligonucleotide array and flowing fluid sorting.

46. A method for detecting nucleotide sequence variation of a target nucleic acid relative to that of a reference nucleic acid, comprising the steps of:
contacting a target nucleic acid with a reference nucleic acid, the sequence of the reference nucleic acid being the same or differing from the target nucleic acid in one or more nucleotide positions;
subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions;
forming a first complex between the Holliday junction structure and a Holliday junction-binder;
contacting the first complex with a receptor for the Holliday junction-binder that specifically recognizes the Holliday junction-binder;
forming a second complex between the first complex and the receptor for the Holliday junction-binder;
labeling one or more strand of the target or reference nucleic acid in the second complex with a tag; and
detecting the presence of the tag on the Holliday junction structure in the second complex.

47. The method of claim 46, wherein the tag is selected from the group consisting of biotin, digoxigenin, fluorescent molecule, chemiluminescent moiety, coenzyme, enzyme substrate, radio isotopes, a particle, nucleic acid-binding protein, and polynucleotide.

48. A method for detecting nucleotide sequence variation of a target nucleic acid relative to that of a reference nucleic acid, comprising the steps of:
contacting a target nucleic acid labeled with a tag with a reference nucleic acid, the sequence of the reference nucleic acid being the same or differing from the target nucleic acid in one or more nucleotide positions;
subjecting a mixture of the target nucleic acid and the reference nucleic acid to branch migration condition such that a Holliday junction structure forms between the target nucleic acid and the reference nucleic acid when the reference nucleic acid differs in sequence from the target nucleic acid in one or more nucleotide positions;
forming a first complex between the Holliday junction structure and a protein that specifically recognizes a Holliday junction;
contacting the first complex with an antibody that specifically binds to the protein that specifically recognizes a Holliday junction;
forming a second complex between the first complex and the antibody; and
detecting the presence of the tag on the target nucleic acid in the Holliday junction structure in the second complex, wherein the presence of the tag on the target nucleic acid in the Holliday junction structure in the second complex is indicative of the sequence difference between the target nucleic acid and the reference nucleic acid.

49. The method of claim 48, wherein the target nucleic acid is derived from a targeted region of a test nucleic acid contained in a sample.

50. The method of claim 49, wherein the target nucleic acid is PCR-amplified from a region of the test nucleic acid containing a SNP.

51. The method of claim 49, wherein the test nucleic acid is selected from the group consisting of double-stranded test DNA, single-stranded test DNA, test RNA, test DNA-RNA hybrid of a target gene, chromosome, plasmid, or genome of a biological material.

52. The method of claim 51, wherein the biological material is selected from the group consisting of bacteria, yeasts, viruses, viroids, molds, fungi, plants, animals, and humans.

53. The method of claim 49, wherein the sequence of the reference nucleic acid differs from the sequence of the target nucleic acid in a single nucleotide position.

54. The method of claim 48, wherein the target nucleic acid is double-stranded.

55. The method of claim 49, wherein the target nucleic acid comprises a combination of Target-Tail-1 polynucleotide and Target-Tail-2 polynucleotide, wherein the Target-Tail-1 polynucleotide comprises a first region that is substantially homologous to the target region of the test nucleic acid and a second region designated as Tail-1; the Target-Tail-2 polynucleotide comprises a first region that is substantially homologous to the target region of the test nucleic acid and a second region designated as Tail-2; and the sequence of Tail-1 and that of Tail-2 differ in one or more nucleotide positions.

56. The method of claim 55, wherein the reference nucleic acid comprises a combination of Reference-Tail-1 polynucleotide and Reference-Tail-2 polynucleotide, wherein the Reference-Tail-1 polynucleotide comprises a first region that is substantially homologous to the target region of the test nucleic acid and a second region designated as Tail-1; the Reference-Tail-2 polynucleotide comprises a first region that is substantially homologous to the target region of the test nucleic acid and a second region designated as Tail-2; and the sequence of Tail-1 and that of Tail-2 differ in one or more nucleotide positions.

57. The method of claim 56, wherein the Target-Tail-1 polynucleotide or the Target-Tail-2 polynucleotide is double-stranded.

58. The method of claim 57, wherein the Reference-Tail-1 polynucleotide or the Reference-Tail-2 polynucleotide is double-stranded.

59. The method of claim 58, further comprising:
PCR-amplifying the targeted region of the test nucleic acid using
a forward primer for the targeted region of the test nucleic acid,
a first reverse primer for the targeted region of the test nucleic acid further comprising the sequence of Tail-1, and
a second reverse primer for the targeted region of the test nucleic acid further comprising the sequence of Tail-2.

60. The method of claim 48, wherein the reference nucleic acid or the target nucleic acid has a length of 20-500 nucleotides.

61. The method of claim 48, wherein the reference nucleic acid or the target nucleic acid has a length of 30-100 nucleotides.

62. The method of claim 48, wherein the reference nucleic acid or the target nucleic acid has a length of 50-80 nucleotides.

63. The method of claim 48, wherein the step of detecting the presence of the Holliday junction structure in a second complex includes detecting the presence of one or more strands of the Holliday junction by a method selected from the group consisting of colorimetric detection, fluorescence detection, chemiluminescent detection, enzymatic reaction, gel electrophoresis, mass spectroscopy, and oligonucleotide array.

64. The method of claim 48, wherein the tag is selected from the group consisting of biotin, digoxigenin, fluorescent molecule, chemiluminescent moiety, coenzyme, enzyme substrate, radio isotopes, a particle, nucleic acid-binding protein, and polynucleotide.

65. The method of claim 48, wherein the antibody that specifically binds to the protein that specifically recognizes a Holliday junction is immobilized to a solid support.

66. The method of claim 65, wherein the solid support is a well of a microplate.

67. The method of claim 65, wherein the microplate is a 96-well plate.

68. The method of claim 48, wherein the tag is biotin and the method further comprises:
contacting the second complex with an agent that comprises streptavidin conjugated to an enzyme.

69. The method of claim 68, wherein the enzyme is selected from the group consisting of alkaline phosphatase, perioxidase, and urease.

70. The method of claim 48, wherein the protein that specifically recognizes a Holliday junction is selected from the group consisting of RuvA, RuvC, RuvB, RusA, RuvG, Cce1 and spCcel from yeast, and Hjc from Pyrococcus furiosusa.

71. The method of claim 48, wherein the protein that specifically recognizes a Holliday junction is a resolvase or recombinase.

72. The method of claim 48, wherein the protein that specifically recognizes a Holliday junction is a recombinant resolvase or recombinase conjugated or fused with a His-tag.

73. The method of claim 48, wherein the antibody is a monoclonal, polyclonal, Fab, fragments of the variable regions, single-chain antibody, or antibody contained in anti-serum.

74. A kit for detecting nucleotide sequence variation of a target nucleic acid relative to that of a reference nucleic acid, the kit comprising:
a reference nucleic acid;
forward and reverse target primers for amplifying a targeted region in a test nucleic acid to generate a target nucleic acid;
a Holliday junction-binder; and
a receptor for the Holliday junction-binder.

75. The kit of claim 74, further comprising:
instructions for how to use the kit to detect mutation or nucleotide variation in a sample containing the test nucleic acid.

76. The kit of claim 74, wherein the receptor for the Holliday junction-binder is an antibody that is attached to a solid support.

77. The kit of claim 76, wherein the solid support is a bead, a well of a culture plate, or a membrane.

78. The kit of claim 74, wherein the target nucleic acid or reference nucleic acid is labeled with biotin, and the kit further comprises streptavidin conjugated to an enzyme.

79. The kit of claim 78, wherein the enzyme is selected from the group consisting of alkaline phosphatase, perioxidase, and urease.

80. The kit off claim 74, wherein the reference nucleic acid comprises a combination of Reference-Tail-1 polynucleotide and Reference-Tail-2 polynucleotide, wherein the Reference-Tail-1 polynucleotide comprises a first region that is substantially homologous to the target region of the test nucleic acid and a second region designated as Tail-1; the Reference-Tail-2 polynucleotide comprises a first region that is substantially homologous to the target region of the test nucleic acid and a second region designated as Tail-2; and the sequence of Tail-1 and that of Tail-2 differ in one or more nucleotide positions.

81. The kit of claim 80, wherein the Reference-Tail-1 polynucleotide or the Reference-Tail-2 polynucleotide is double-stranded.

82. The kit of claim 80, wherein the Reference-Tail-1 polynucleotide or the Reference-Tail-2 polynucleotide is single-stranded.

83. The kit of claim 80, wherein the reverse primer comprises a combination of:
a first reverse primer for the targeted region of the test nucleic acid further comprising the sequence of Tail-1, and
a second reverse primer for the targeted region of the test nucleic acid further comprising the sequence of Tail-2.
